(19) United States
(12) Patent Application Publication
(10) Pub. No.: US 2015/0125950 A1
(43) Pub. Date: May 7, 2015

(54) UMBILICAL CORD MESENCHYMAL STEM CELL EXOSOMES

(71) Applicants: AGENCY FOR SCIENCE, TECHNOLOGY AND RESEARCH (A*STAR), Singapore (SG); SINGAPORE HEALTH SERVICES PTE LTD, Singaport (SG)

(72) Inventors: Sai Kiang Lim, Singapore (SG); Kok Hian Tan, Singapore (SG)

(73) Assignee: AGENCY FOR SCIENCE, TECHNOLOGY AND RESEARCH (A*STA (A*STAR), Singapore (SG)

(21) Appl. No.: 14/401,872

(22) PCT Filed: May 17, 2013
(86) PCT No.: PCT/SG2013/000203

§ 371 Date: Nov. 18, 2014

Related U.S. Application Data
(60) Provisional application No. 61/649,132, filed on May 18, 2012.

(51) Int. Cl. C12N 5/0775

(52) U.S. Cl. CPC C12N 5/0668
USPC 435/325

(57) ABSTRACT

We describe a method of cell culture of an umbilical mesenchymal stem cell (MSC). The method comprises the steps of providing an umbilical mesenchymal stem cell (MSC) and culturing the umbilical mesenchymal stem cell in a cell culture medium under hypoxic conditions. The umbilical mesenchymal stem cell may be transformed with an oncogene such as c-myc. Conditioned media and exosomes obtained from the umbilical mesenchymal stem cell may be used for therapy.

### FIELD

The present invention relates to the fields of medicine, cell biology, molecular biology and genetics. This invention relates to the field of medicine. More particularly, the invention relates to mesenchymal stem cells derived from umbilical stem cells and conditioned medium and exosomes produced by such cells.

### BACKGROUND

Mesenchymal stem cells (MSCs) are multipotent stem cells that have a limited but robust potential to differentiate into mesenchymal cell types, e.g. adipocytes, chondrocytes and osteocytes, with negligible risk of teratoma formation. MSC transplantation has been used to treat musculoskeletal injuries, improve cardiac function in cardiovascular disease and ameliorate the severity of graft-versus-host-disease¹. In recent years, MSC transplantations have demonstrated therapeutic efficacy in treating different diseases but the underlying mechanism has been controversial²⁻⁹. Some reports have suggested that factors secreted by MSCs¹⁰ were responsible for the therapeutic effect on arteriogenesis¹¹, stem cell crypt in the intestine¹², ischemic^{9,13-18}, and hematopoiesis^{19,20}. In support of this paracrine hypothesis, many studies have observed that MSCs secrete cytokines, chemokines and growth factors that could potentially repair injured cardiac tissue mainly through cardiac and vascular tissue growth and regeneration^{21,22}. This paracrine hypothesis could potentially provide for a non-cell based alternative for using MSC in treatment of cardiovascular disease²³. Non-cell based therapies as opposed to cell-based therapies are generally easier to manufacture and are safer as they are non-viable and do not elicit immune rejection.

We have previously demonstrated that culture medium conditioned by mesenchymal stem cells (MSCs) that were derived from human embryonic stem cells or fetal tissues²⁴⁻²⁶ could protect the heart from myocardial ischemia/reperfusion injury and reduce infarct size in both pig and mouse models of MI/R injury²⁵⁻²⁷. Subsequent studies demonstrated that this cardioprotection was mediated by exosomes or microparticles of about 50-100 ηm in diameter and these microparticles carry both protein and RNA load²⁴⁻²⁸. These exosomes could be purified as a population of homogenously sized particles by size exclusion on HPLC^{25,26} and reduced infarct size in a mouse model of MI/R injury at about a tenth of the dosage of the conditioned medium.

The identification of exosomes as the therapeutic agent in the MSC secretion could potentially provides for a biologic—rather than cell-based treatment modality. Unlike cells, exosomes do not elicit acute immune rejection and being non-viable and much smaller. They pose less safety risks such as the formation of tumor or embolism. Unlike cell-based therapies where there is a need to maintain viability, manufacture and storage of non-viable exosomes is less complex and therefore less costly. Besides being therapeutic agents, exosomes have been advocated as “natural” drug delivery vehicles²⁹. These lipid vesicles could be loaded with therapeutic agents and be used to deliver the agents in a cell type specific manner. hESC-MSCs could be the ideal cellular source for the efficient production of exosomes. We have demonstrated that these cells could be grown in a chemically defined during the production and harvest of exosomes and these exosomes could be purified by HPLC to generate a population of homogenously sized particles²⁵⁻²⁷. Another advantage is that these cells were derived from hESC, an infinitely expansible cell source.

While hESC-MSCs are also highly expansible in culture, they unlike their parental hESC can undergo only a finite number of cell divisions before their growth is arrested and they senesce. Therefore there will be a need to constantly derive new batches of MSCs from hESCs to replenish the cell source of exosomes with each derivation necessitating recurring cost of derivation, testing and validation. To circumvent this need for re-derivation and ensure an infinite supply of identical MSCs for commercially sustainable production of exosomes as therapeutic agents or delivery vehicle, we have previously immortalized hESC-MSC with c-myc³⁰. Although it was previously reported that transfection of v-myc gene into fetal MSCs immortalized the cells but did not alter the fundament characteristics of these MSCs³¹, we observed that transformed hESC-MSCs have some differences from their parental cells. They have reduced plastic adherence, faster growth, failure to senesce, increased myc expression and loss of in vitro adipogenic potential that technically rendered the transformed cells as non-MSCs. However, they also retained many of their parental cell characteristics, such as a surface antigen profile of CD29⁺, CD44⁺, CD49a⁺ CD49e⁺, CD105⁺, CD166⁺, MHCI⁺, CD34⁻, CD45⁻ and HLA-DR⁻. Like their parental cells, they also secreted exosomes that were able to reduce relative infarct size in a mouse model of myocardial ischemia/reperfusion injury.

MSCs from ethically palatable adult tissue sources such as cord, bone marrow and adipose tissues are generally not amenable to the production of exosomes as their ex vivo expansion capacity is much smaller than that of hESC-derived MSCs.

### SUMMARY

According to a 1^{st} aspect of the present invention, we provide a method of cell culture of an umbilical mesenchymal stem cell (MSC). The method comprises the steps of providing an umbilical mesenchymal stem cell (MSC) and culturing the umbilical mesenchymal stem cell in a cell culture medium under hypoxic conditions.

The umbilical mesenchymal stem cell may comprise a transformed umbilical mesenchymal stem cell.

The method may be such that an oncogene such as c-myc has been introduced into the umbilical mesenchymal stem cell to thereby transform it. The oncogene may be introduced into an ancestor of the umbilical mesenchymal stem cell.

The method may be such that the hypoxic conditions comprise 10% or less oxygen. The hypoxic conditions may comprise 5% or less oxygen. The hypoxic conditions may comprise about 1% oxygen.

The method may comprise culturing the umbilical mesenchymal stem cell in a cell culture medium to condition it. The method may comprise separating the cell culture medium from the umbilical mesenchymal stern cell.

The method may comprise producing a umbilical mesenchymal stem cell conditioned medium (MSC-CM).

The method may further comprise isolating an exosome from the cell culture medium.

The method may comprise obtaining an umbilical mesenchymal stem cell conditioned medium (MSC-CM) by a method set out above. The method may comprise concentrating the umbilical mesenchymal stem cell conditioned medium, for example by ultrafiltration over a >1000 kDa membrane. The method may comprise subjecting the concentrated umbilical mesenchymal stem cell conditioned medium to size exclusion chromatography. The method may use a TSK Guard column SWXL, 6×40 mm or a TSK gel G4000 SWXL or a 7.8×300 mm column. The method may comprise selecting UV absorbent fractions that exhibit dynamic light scattering, The UV absorbent fractions may absorb at 220 nm. The method may comprise detection by a quasi-elastic light scattering (QELS). The method may comprise collecting fractions which elute with a retention time of 11-13 minutes, such as 12 minutes.

The method may be such that an umbilical mesenchymal stem cell cultured under hypoxic conditions is capable of producing 150% or more exosomes than a umbilical mesenchymal stem cell cultured under normoxic conditions.

The method may be such that an umbilical mesenchymal stem cell cultured under hypoxic conditions is capable of producing 200% or more

The method may be such that an umbilical mesenchymal stem cell cultured under hypoxic conditions is capable of producing 250% or more exosomes than a umbilical mesenchymal stem cell cultured under normoxic conditions.

The method may be such that an umbilical mesenchymal stem cell cultured under hypoxic conditions is capable of producing 300% or more exosomes than a umbilical mesenchymal stem cell cultured under normoxic conditions.

There is provided, according to a 2^{nd} aspect of the present invention, a method of preparing a pharmaceutical composition. The method may comprise obtaining a umbilical mesenchymal stem cell conditioned medium (MSC-CM) or an exosome by a method set out above. The umbilical mesenchymal stem cell conditioned medium or exosome so obtained may be admixed with a pharmaceutically acceptable carrier or diluent.

The method may be such that the umbilical mesenchymal stem cell, the conditioned medium, exosome or pharmaceutical composition comprises at least one biological property of a umbilical mesenchymal stem cell. The biological property may comprise cardioprotection.

The conditioned medium, exosome or pharmaceutical composition may be capable of reducing infarct size. The reduction of infarct size may be assayed in a mouse or pig model of myocardial ischemia and reperfusion injury.

The conditioned medium, exosome or pharmaceutical composition may be capable of reducing oxidative stress. The reduction of oxidative stress may be assayed in an in vitro assay of hydrogen peroxide (H₂O₂)-induced cell death.

We provide, according to a 3^{rd} aspect of the present invention, an umbilical mesenchymal stem cell for use in a method of treatment of a disease in an individual. The method may comprise culturing an umbilical mesenchymal stem cell as described. The method may comprise obtaining an umbilical mesenchymal stem cell conditioned medium (MSC-CM) or an exosome therefrom. The method may comprise administering the umbilical mesenchymal stem cell conditioned medium (MSC-CM) or the exosome into an individual in need of treatment.

The umbilical mesenchymal stem cell conditioned medium may be obtained by a method set out above. The exosome may be obtained by a method set out above.

As a 4^{th} aspect of the present invention, there is provided an umbilical mesenchymal stem cell conditioned medium or an exosome for use in a method of treatment of a disease in an individual. The method may comprise obtaining an umbilical mesenchymal stem cell conditioned medium (MSC-CM) as set out above. The method may comprise obtaining an exosome as described. The method may comprise administering the umbilical mesenchymal stem cell conditioned medium (MSC-CM) or the exosome into an individual in need of treatment.

We provide, according to a 5^{th} aspect of the present invention, an umbilical mesenchymal stem cell obtainable from an umbilical tissue such as umbilical cord, a descendent of such an umbilical mesenchymal stem cell, a cell culture or a cell line comprising either, which is or has been cultured under hypoxic conditions.

The umbilical mesenchymal stem cell, descendent, cell culture or cell line may be such that culture under hypoxic conditions enables the umbilical mesenchymal stem cell, descendent, cell culture or cell line to produce 150% or more exosomes than an umbilical mesenchymal stem cell, descendent, cell culture or cell line cultured under normoxic conditions. It may produce 200% or more, 250% or more, or 300% or more exosomes than if cultured under normoxic conditions.

The umbilical mesenchymal stem cell, descendent, cell culture or cell line may be one, into which, or into an ancestor of which, an oncogene such as c-myc has been introduced to thereby transform it.

In a 7^{th} aspect of the present invention, there is provided a conditioned medium obtainable from an umbilical mesenchymal stem cell, descendent, cell culture or cell line as described.

According to an 8^{th} aspect of the present invention, we provide an exosome obtainable from a conditioned medium as set out above.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 is a diagram showing karyotype of CMSC3A1 by G-banding

FIG. 2 is a diagram showing relative Myc transcript level. Myc transcripts level in HuES9.E1 (MSCs derived from hESC), E1-myc 16.3 (myc transformed HuES9.E1 MSC line) at p8 and p18, and CMSC3A1 myc-transformed cord MSC line at p8 and p18 were determined by quantitative RT-PCR. The internal reference for each sample was GAPDH transcript. The myc transcript level in each sample was normalized to that in HuES9.E1.

FIG. 3 is a diagram showing rate of cell cycling. Cells were labelled with CFDA and their fluorescence was monitored over time by flow cytometry. The loss of cellular fluorescence at each time point was used to calculate the number of cell division that the cells have undergone as described in Materials and Methods

FIG. 4 is a diagram showing cell morphology of cord and myc-immortalized cord MSC line, CMSC3A1 as observed under light microscopy.

FIG. 5 is a diagram showing relative telomerase activity. Telomerase activity in each cell type was assayed using 1 μg of cell lysate protein to first extend a TS primer and any extended product was then quantitated by real time PCR. The Ct value reflected the amount of telomerase product and therefore the telomerase activity in the lysate. Note: Ct value is inversely proportional to the template concentration in the PCR reaction.

FIG. 6 is a diagram showing surface antigen profile. CMSC3A1 (green) and HuES9.E1 (red) MSCs were stained with a specific antibody conjugated to a fluorescent dye and analyzed by FACS. Nonspecific fluorescence (purple) was assessed by incubating the cells with isotype-matched mouse monoclonal. antibodies.

FIG. 7 is a diagram showing differentiation of CMSC3A1. MSCs were induced to undergo a) osteogenesis and then stained with von Kossa stain; b) chondrogenesis and then stained with Alcian blue; c) adipogenesis where CMSC3A1 and. HuES9.E1 MSCs were exposed to adipogenesis induction medium for two days.

FIG. 8 is a diagram showing gene expression profile of MSCs and their myc-transformed progenies. A heat map of the gene expression profile of the different MSCs and their myc-transformed progenies at different passage numbers.

FIG. 9 is a diagram showing HPLC fractionation of CMSC3A1 conditioned medium. CMSC3A1 conditioned medium was fractionated on a HPLC using BioSep 54000, 7.8 mm×30 cm column. The components in CM were eluted with 20 mM phosphate buffer with 150 mM of NaCl at pH 7.2. The elution mode was isocratic and the run time was 40 minutes. The eluent was monitored for UV absorbance at 220 ηm. Each eluting peak was then analyzed by light scattering. The fastest eluting peak was collected for testing in a mouse model of ischemia/reperfusion injury.

FIG. 10 is a diagram showing exosome production during normoxia and hypoxia The relative level of exosomes in the conditioned medium was determined by extracting exosomes and measuring CD9 protein level in the extract. Exosomes were extracted by cholera toxin B chain affinity chromatography and CD9 in the extracts was measured by ELISA using CD9 specific antibody.

FIG. 11 is a diagram showing cardioprotection by CMSC3A1 exosomes. 0.3 μg HPLC-purified exosomes from either or E1-myc 16.3 or CMSC3A1 was administered intravenously to a mouse model of acute myocardial/ischemia reperfusion injury five minutes before reperfusion. Infarct size (IS) as a percentage of the area at risk (AAR) upon treatment with saline (n=10), E1myc 16.3 exosomes (n=4) and CMSC3A1 (n=4) were measured. The relative infarct size (IS/AAR) in mice treated with E1-myc 16.3 and CMSC3A1 exosome was 22.6±4.5%, and 19.8±2.9%, respectively. These relative infarct sizes were significantly lower than the relative infarct size of 38.5±5.6% in saline-treated mice (p<0.002 and p<0.001, respectively).

FIG. 12 is a diagram showing a difference between exosomes secreted by Myc-immortalized cord MSCs from those secreted by ESC-derived MSCs.

### DETAILED DESCRIPTION

We describe a mesenchymal stem cell which is derivable from an umbilical cell. We also describe methods of culture of such cells.

The methods of cell culture described here may make use of hypoxic cell culture. Thus, for example, we describe a method of culturing umbilical mesenchymal stem cells, which may be transformed, under hypoxic conditions. We demonstrate that such cell culture enables the umbilical mesenchymal stem cells to produce higher levels of exosomes than under normoxic culture.

Such an umbilical mesenchymal stem cell may be referred to in this document as an “umbilical mesenchymal stem cell” or a mesenchymal stem cell obtained by the methods described in this document.

The umbilical cell from which the mesenchymal stem cell is derived may comprise any cell from the umbilicus of a mammal, such as an umbilical cord blood cell, an umbilical vein subendothelium cell, an umbilical cord Wharton's Jelly cell or a subamnion cell. Accordingly, the umbilical mesenchymal stem cells described in this document may comprise umbilical cord blood derived mesenchymal stem cells, umbilical vein subendothelium derived mesenchymal stem cells, umbilical cord Wharton's Jelly derived mesenchymal stem cells or subamnion derived mesenchymal stem cells.

The umbilical mesenchymal stem cell may comprise a mammalian, primate or human umbilical mesenchymal stem cell.

The umbilical mesenchymal stem cell may comprise a suitable cell surface antigen profile, such as one or more (for example all of) CD29⁺, CD44⁺, CD49a⁺ CD49e⁺, CD73⁺ CD105⁺, CD166⁺, MHC I⁻, HLA-DR⁻, CD34⁻ and CD45⁻.

The umbilical mesenchymal stem cell may be in the form of a cell, cell culture or cell lined derived therefrom. Methods of deriving cell cultures and cell lines are well known in the art. The cell cultures and/or cell lines derived from or comprising umbilical mesenchymal stem cells described here may comprise any one or more, such as all, features ascribed to the umbilical mesenchymal stem cell.

The umbilical mesenchymal stem cell may comprise a normal karyotype appropriate to the species from which it is derived. For example, where the umbilical mesenchymal stem cell comprises a human umbilical mesenchymal stem cell, the karyotype may comprise a normal 46 XY karyotype.

The umbilical mesenchymal stem cell may have differentiation potential, such as being multipotent. The umbilical mesenchymal stem cell may be capable of differentiating into chondrocytes. It may be capable of differentiating into osteocytes. It may be such that it is not capable of differentiating into adipocytes.

The umbilical mesenchymal stem cell may comprise a transformed umbilical mesenchymal stem cell, as described in further detail below. Unless the context dictates otherwise, the term “umbilical mesenchymal stem cell” should be understood to include transformed umbilical mesenchymal stem cells. Accordingly, the properties and uses ascribed to umbilical mesenchymal stem cells should also be taken as being applicable to transformed umbilical mesenchymal stem cells.

The transformed umbilical mesenchymal stem cell may be transformed by any suitable means, such as by introduction of an oncogene into the cell, or an ancestor of the cell. The oncogene may comprise for example c-myc. The cell cycle time of such a transformed umbilical mesenchymal stem cell may be shorter compared to an untransformed umbilical mesenchymal stem cell. The transformed umbilical mesenchymal stem cell smaller, rounder and/or have more prominent nuclei; they may have reduced adherence to plastic culture and/or reduced contact inhibition at confluency. In particular, the transformed umbilical mesenchymal stem cell may be more prone to forming clusters instead of adhering to the plastic dish as a monolayer, when compared to non-transformed umbilical mesenchymal stem cells. They may comprise higher telomerase activity, as well.

The umbilical mesenchymal stem cell line may comprise a CMSC3A1, CMSC3A2 or CMSC3A3 cell line.

The umbilical mesenchymal stem cell, cell culture, cell line etc may be cultured under hypoxic conditions.

We further provide a medium which is conditioned by culture, preferably hypoxic culture, of the umbilical mesenchymal stem cells. Such a conditioned medium is referred to in this document as a “umbilical mesenchymal stem cell conditioned medium” and is described in further detail below. The term “umbilical mesenchymal stem cell conditioned medium” should be understood as encompassing medium conditioned by any umbilical mesenchymal stem cells described in this document, including transformed umbilical mesenchymal stem cells.

We further provide a particle secreted by a umbilical mesenchymal stem cell and comprising at least one biological property of a umbilical mesenchymal stem cell. We refer to such a particle in this document as a “umbilical mesenchymal stem cell particle”. The term “umbilical mesenchymal stem cell particle” includes particles derived from any umbilical mesenchymal stem cells described in this document, including transformed umbilical mesenchymal stem cells.

Such a particle is described in further detail below, and a summary follows.

The biological property of the umbilical mesenchymal stem cell particle may comprise a biological activity of a umbilical mesenchymal stem cell conditioned medium (MSC-CM). The biological activity may comprise cardioprotection.

The umbilical mesenchymal stem cell particle may be capable of reducing infarct size. Reduction of infarct may be assayed in a mouse or pig model of myocardial ischemia and reperfusion injury.

The umbilical mesenchymal stem cell particle may be capable of reducing oxidative stress. The reduction of oxidative stress may be assayed in an in vitro assay of hydrogen peroxide (H₂O₂)-induced cell death.

The umbilical mesenchymal stem cell particle may comprise a vesicle. The umbilical mesenchymal stem cell particle may comprise an exosome. The umbilical mesenchymal stem cell particle may contain at least 70% of proteins in an umbilical mesenchymal stem cell conditioned medium (MSC-CM).

The umbilical mesenchymal stem cell particle may comprise a complex of molecular weight >100 kDa. The complex of molecular weight >100 kDa may comprise proteins of <100 kDa. The particle may comprise a complex of molecular weight >300 kDa. The complex of molecular weight >100 kDa may comprise proteins of <300 kDa.

The umbilical mesenchymal stem cell particle may comprise a complex of molecular weight >1000 kDa. The particle may have a size of between 2 nm and 200 nm. The umbilical mesenchymal stem cell particle may have a size of between 50 ηm and 150 nm. The umbilical mesenchymal stem cell-particle may have a size of between between 50 nm and 100 nm.

The size of the umbilical mesenchymal stem cell particle may be determined by filtration against a 0.2 μM filter and concentration against a membrane with a molecular weight cut-off of 10 kDa. The size of the umbilical mesenchymal stem cell particle may be determined by electron microscopy.

The umbilical mesenchymal stem cell particle may comprise a hydrodynamic radius of below 100 nm. It may comprise a hydrodynamic radius of between about 30 nm and about 70 nm. It may be between about 40 nm and about 60 nm, such as between about 45 nm and about 55 nm. The umbilical mesenchymal stem cell particle may comprise a hydrodynamic radius of about 50 nm. The hydrodynamic radius may be determined by laser diffraction or dynamic light scattering.

The umbilical mesenchymal stem cell particle may comprise a lipid selected from the group consisting of: phospholipid, phosphatidyl serine, phosphatidyl inositol, phosphatidyl choline, shingomyelin, ceramides, glycolipid, cerebroside, steroids, cholesterol. The cholesterol-phospholipid ratio may be greater than 0.3-0.4 (mol/mol). The umbilical mesenchymal stem cell particle may comprise a lipid raft.

The umbilical mesenchymal stem cell particle may be insoluble in non-ionic detergent, preferably Triton-X100. The umbilical mesenchymal stem cell particle may be such that proteins of the molecular weights specified substantially remain in the complexes of the molecular weights specified, when the umbilical mesenchymal stem cell particle is treated with a non-ionic detergent.

The umbilical mesenchymal stem cell particle may be sensitive to cyclodextrin, preferably 20 mM cyclodextrin. The umbilical mesenchymal stem cell particle may be such that treatment with cyclodextrin causes substantial dissolution of the complexes specified.

The umbilical mesenchymal stem cell particle may comprise ribonucleic acid (RNA). The particle may have an absorbance ratio of 1.9 (260:280 nm). The umbilical mesenchymal stem cell particle may comprise a surface antigen selected from the group consisting of: CD9, CD109 and thy-1.

We further describe a method of producing a umbilical mesenchymal stem cell particle as described above, the method comprising isolating the umbilical mesenchymal stem cell particle from a umbilical mesenchymal stem cell conditioned medium (MSC-CM).

The method may comprise separating the umbilical mesenchymal stem cell particle from other components based on molecular weight, size, shape, composition or biological activity.

The weight may be selected from the weights set out above. The size may be selected from the sizes set out above. The composition may be selected from the compositions set out above. The biological activity may be selected from the biological activities set out above.

We further describe a method of producing a umbilical mesenchymal stem cell particle as described above. The method may comprise obtaining a umbilical mesenchymal stem cell conditioned medium (MSC-CM). It may comprise concentrating the umbilical mesenchymal stem cell conditioned medium. The umbilical mesenchymal stem cell conditioned medium may be concentrated by ultrafiltration over a >1000 kDa membrane. The method may comprise subjecting the concentrated umbilical mesenchymal stem cell conditioned medium to size exclusion chromatography. A TSK Guard column SWXL, 6×40 mm or a TSK gel G4000 SWXL, 7.8×300 mm column may be employed. The method may comprise selecting UV absorbent fractions, for example, at 220 nm, that exhibit dynamic light scattering. The dynamic light scattering may be detected by a quasi-elastic light scattering (QELS) detector. The method may comprise collecting fractions which elute with a retention time of 11-13 minutes, such as 12 minutes.

We further provide a pharmaceutical composition comprising a umbilical mesenchymal stem cell particle as described together with a pharmaceutically acceptable excipient, diluent or carrier.

We further provide such a umbilical mesenchymal stem cell particle or such a pharmaceutical composition for use in a method of treating a disease.

We further provide for the use of such an umbilical mesenchymal stem cell particle for the preparation of a pharmaceutical composition for the treatment of a disease.

We further provide for the use of such a umbilical mesenchymal stem cell particle in a method of treatment of a disease in an individual.

The disease may be selected from the group consisting of: cardiac failure, bone marrow disease, skin disease, burns and degenerative diseases such as diabetes, Alzheimer's disease, Parkinson's disease and cancer.

The disease may be selected from the group consisting of: myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

The umbilical mesenchymal stem cell particle may be used to aid wound healing, scar reduction, bone formation, a bone graft or bone marrow transplantation in an individual.

The umbilical mesenchymal stem cell particle may be used (i) in the regulation of a pathway selected from any one or more of the following: cytoskeletal regulation by Rho GTPase, cell cycle, integrin signaling pathway, Inflammation mediated by chemokine & cytokine signaling pathway, FGF signaling pathway, EGF receptor signaling pathway, angiogenesis, plasminogen activating cascade, blood coagulation, glycolysis, ubiquitin proteasome pathway, de novo purine biosynthesis, TCA cycle, phenylalanine biosynthesis, heme biosynthesis; (ii) in the regulation of processes including any one or more of the following: cell structure and motility, cell structure, cell communication, cell motility, cell adhesion, endocytosis, mitosis, exocytosis, cytokinesis, cell cycle, immunity and defense, cytokine/chemokine mediated immunity, macrophage-mediated immunity, granulocyte-mediated immunity, ligand-mediated signaling, cytokine and chemokine mediated signaling pathway, signal transduction, extracellular matrix protein-mediated signaling, growth factor homeostasis, receptor protein tyrosine kinase signaling pathway, cell adhesion-mediated signaling, cell surface receptor mediated signal transduction, JAK-STAT cascade, antioxidation and free radical removal, homeostasis, stress response, blood clotting, developmental processes, mesoderm development, skeletal development, angiogenesis, muscle development, muscle contraction, protein metabolism and modification, proteolysis, protein folding, protein complex assembly, amino acid activation, intracellular protein traffic, other protein targeting and localization, amino acid metabolism, protein biosynthesis, protein disulfide-isomerase reaction, carbohydrate metabolism, glycolysis, pentose-phosphate shunt, other polysaccharide metabolism, purine metabolism, regulation of phosphate metabolism, vitamin metabolism, amino acid biosynthesis, pre-mRNA processing, translational regulation, mRNA splicing; or (iii) in the supply of functions including any one or more of the following: signaling molecule, chemokine, growth factor, cytokine, interleukin, other cytokine, extracellular matrix, extracellular matrix structural protein, other extracellular matrix, extracellular matrix glycoprotein, protease, metalloprotease, other proteases, protease inhibitor, metalloprotease inhibitor, serine protease inhibitor, oxidoreductase, dehydrogenase, peroxidase, chaperone, chaperonin, Hsp 70 family chaperone, other chaperones, synthetase, synthase and synthetase, select calcium binding protein, aminoacyl-tRNA synthetase, lyase, isomerase, other isomerase, ATP synthase, hydratase, transaminase, other lyase, other enzyme regulator, select regulatory molecule, actin binding cytoskeletal protein, cytoskeletal protein, non-motor actin binding protein, actin and actin related protein, annexin, tubulin, cell adhesion molecule, actin binding motor protein, intermediate filament, ribonucleoprotein, ribosomal protein, translation factor, other RNA-binding protein, histone, calmodulin related protein, vesicle coat protein.

We further provide for a delivery system for delivering a umbilical mesenchymal stem cell particle, comprising a source of umbilical mesenchymal stem cell particle together with a dispenser operable to deliver the particle to a target.

We further provide use of such a delivery system in a method of delivering a particle to a target.

We demonstrate in the Examples that umbilical mesenchymal stem cells mediate cardioprotective effects through secreted complexes such as exosomes. Such complexes or particles may therefore be used for therapeutic means, including for cardioprotection, in place of the cells themselves.

The umbilical mesenchymal stem cell particles, complexes or exosomes may be used for a variety of purposes, such as treatment or prevention for cardiac or heart diseases such as ischaemia, cardiac inflammation or heart failure. They may also be used for repair following perfusion injury.

### Culture of Umbilical Mesenchymal Stem Cells Under Hypoxic Conditions

The term “hypoxic culture” as it is used in this document is meant to refer to a cell culture protocol in which, for at least part of the time, the cultured cells are exposed to hypoxic conditions in which oxygen is reduced compared to normal oxygen conditions.

Normal oxygen conditions are referred to as “normoxia”, and for the purposes of this document, may be taken to mean about 21% oxygen, for example about 20.9% oxygen. Under normoxia, other gases may be present, such as 78% nitrogen and 1% other gases (including noble gases such as argon).

Hypoxic conditions may comprise any concentration of oxygen below 21%, for 15% oxygen or less, 14% oxygen or less, 13% oxygen or less, 12% oxygen or less, 11% oxygen or less, 10% oxygen or less, 9% oxygen or less, 8% oxygen or less, 7% oxygen or less, 6% oxygen or less, 5% oxygen or less, 4% oxygen or less, 3% oxygen or less, 2% oxygen or less or 1% oxygen or less. Hypoxic conditions may for example comprise about 1% oxygen.

As measured by pressure, normal levels of oxygen may comprise 135 mmHg, with hypoxic conditions comprising about 30-40 mmHg.

The hypoxic conditions may comprise carbon dioxide, for example at 5%. The remainder of the gas composition may comprise an inert gas such as nitrogen. An example gas composition suitable for hypoxic culture may comprise 1% oxygen, 5% carbon dioxide and 94% nitrogen.

The hypoxic cell culture may take place using suitable cell culture apparatatus, such as a closed incubator and closed hood with a controlled level of oxygen (Xvivo, Biospherix, NY, USA).

The umbilical mesenchymal stem cells, including transformed umbilical mesenchymal stem cells, may be cultured under hypoxic conditions for a time sufficient for the umbilical mesenchymal stem cells to increase production of exosomes or secreted particles. They may be cultured of course for longer than this period.

The increase in production of exosomes may be by 10% or more, 20% or more, 30% or more, 40% or more, 50% or more, 60% or more, 70% or more, 80% or more, 90% or more or 100% or more, as compared to control cells, i.e., such cells cultured under normoxic conditions. They may be cultured for a length of time sufficient for 2 times or more, 2.1 times or more, 2.2 times or more, 2.3 times or more, 2.4 times or more, 2.5 times or more, 2.6 times or more, 2.7 times or more, 2.8 times or more, 2.9 times or more or 3 times or more exosomes than control cells.

The increase in the production of exosomes may be measured by their number or activity, such as by tracking the amount of protein specific to the exosomes that are produced by the cells. For example, the production of exosomes may be measured by extracting exosomes and measuring CD9 protein in the extract, by means known in the art (e.g., immunoassay using anti-CD9 antibodies).

The cell culture of umbilical mesenchymal stem cells may comprise one or more periods of hypoxic as described above. Where there is more than one hypoxic period, the cell culture in between the hypoxic periods may comprise normoxic cell culture. Therefore, the umbilical mesenchymal stem cells may be cultured for example under hypoxic conditions, followed by culture in normoxic conditions. This may be followed by a period under hypoxic conditions, followed optionally by one or more normoxic/hypoxic cycles. The culture may start off with normoxic culture followed by hypoxic culture, followed optionally by one or more normoxic/hypoxic cycles. Various combinations are possible.

The periods under which the cells are cultured under hypoxic conditions and normoxic conditions may be substantially identical or similar in duration, or may vary. The lengths of time may for example comprise 1 to 12 hours or more, such as 18 hours or 24 hours.

### Umbilical Mesenchymal Stem Cells

Umbilical mesenchymal stem cells suitable for use in the methods and compositions described here may come from a variety of sources. Umbilical mesenchymal stem cells are reviewed in Troyer et al (2008) Concise Review: Wharton's Jelly-Derived Cells are a Primitive Stromal Cell Population, Stem Cells 26, 591-599.

Any of the umbilical mesenchymal stem cells described in that document, for example as set out in Table 1 thereof, may be employed in the methods and compositions described here.

Umbilical Cord Blood Derived Mesenchymal Stem Cells

Umbilical mesenchymal stem cells suitable for use in the methods and compositions described here may be derived from umbilical cord blood, including any of the following:

Umbilical cord blood-derived multilineage progenitor cells (MLPC) described in Berger M J, et al., 2006; Mesenchymal stem cell-like cells (MSC-like cells) described in Bieback K, et al., 2004; Mesenchymal-like cells (MLCs) described in Erices A, et al., 2000; Umbilical cord blood-derived mesenchymal stem cells (UCB-derived MSCs) described in Gang E J, et al., 2004; Umbilical cord blood-derived mesenchymal stem cells (UCB-derived MSCs) described in Gang E J, et al., 2006; Umbilical cord blood-derived stromal cell described in Gao L, et al., 2006; Non-hematopoietic progenitors (NHPs) described in 2005; MSC from umbilical cord blood (MSCs) described in Hou L, et al., 2003; Umbilical cord blood-derived mesenchymal stem cells (UCB-derived MSCs) described in Jang Y K, et al., 2006; Human umbilical cord blood-derived mesenchymal stem cells (MSCs) described in Jeong J A, et al., 2005; umbilical mesenchymal stem cells described in Kang X Q, et al., 2006; AC133-CD14+ umbilical cord blood-derived cells (AC133-CD14+ cells) described in Kim S Y, et al., 2005; Umbilical cord blood-derived mesenchymal stem cells (UCB-MSCs) described in Kern S, et al., 2006; Unrestricted somatic stem cells from human cord blood (USSC) described in Kogler G, et al., 2004; Multipotent mesenchymal stem cells from umbilical cord blood described in Lee O K, et al., 2004; Umbilical cord blood-derived mesenchymal stem cells (UCB-MSCs) described in Lu F Z, et al., 2005; Cord blood derived-mesenchymal stem cells (CB-MSC) described in Wagner W, et al., 2005; Human mesenchymal stem/progenitor cell described in Wang J-F, et al., 2004; Human mesenchymal stem/progenitor cell (MSPC) described in Yang S-E, et al., 2004; Human umbilical cord blood stromal cells (HUCB-derived adherent layer cultures) described in Ye ZQ, et al., 1994; Fetal blood derived mesenchymal stem cells described in Yu M, et al., 2004.

Umbilical Vein Subendothelium Derived Mesenchymal Stem Cells

Umbilical mesenchymal stem cells suitable for use in the methods and compositions described here may be derived from umbilical vein subendothelium, including any of the following:

Umbilical vein mesenchymal stem cells described in Covas D T, et al., 2003; Mesenchymal progenitor or stem cells, fibroblastic and endothelial cells isolated described in Kim J W, et al., 2004; Umbilical cord-mesenchymal stem cells (UC-MSCs) described in Panepucci R A, et al., 2004; Mesenchymal stem cell-like cells (MSC-like cells) described in Romanov Y A, et al., 2003; Umbilical fibroblast-like cells described in Yarygin K N, et al, 2006.

Umbilical Cord Perivascular Cell Derived Mesenchymal Stem Cells

Umbilical mesenchymal stem cells suitable for use in the methods and compositions described here may be derived from umbilical cord perivascular cells, including any of the following:

Human umbilical cord perivascular cells (HUCPVCs) described in Baksh K, et al., 2007; Human umbilical cord perivascular cells (HUCPV cells) described in Sarugaser R, et al., 2005.

Umbilical Cord Wharton's Jelly Derived Mesenchymal Stem Cells

Umbilical mesenchymal stem cells suitable for use in the methods and compositions described here may be derived from umbilical cord Wharton's jelly, including the perivascular zone of Wharton's jelly, the intervascular zone of Wharton's jelly, and the subamnion of Wharton's jelly.

Umbilical mesenchymal stem cells suitable for use in the methods and compositions described here may therefore include any of the following:

Human umbilical cord matrix cells (HUCM) described in Bailey M M, et al., 2007; CD105+/CD31−/KDR− cells described in Conconi M T, et al., 2006; Umbilical cord mesenchymal cells described in Fu Y-S, et al., 2004; Human umbilical mesenchymal stem cells (HUMSCs) described in Fu Y-S, et al., 2006; Rat umbilical cord matrix cells (RUCMs) described in Jomura, S, et al., 2006; Umbilical cord cells (UCC) described in Kadner A et al., 2002; Whole umbilical cord (WUCC) described in Kadner A et al., 2004; Human umbilical cord stroma cell, intervascular cells (IVCs) vs perivascular cells (PVCs) (HUCSCs) described in Karahuseyinoglu, S, et al., 2006; Mesenchymal stem cells derived from umbilical cord (UC-MSC) described in Lu L-L, et al, 2006; Human umbilical cord-derived cells (hUTC) described in Lund R D, et al., 2007; Wharton's Jelly mesenchymal stem cells described in Ma L, et al., 2005; Pig umbilical cord matrix' stem cells (UCM cells) described in Medicetty S, et al., 2004; Wharton's jelly cells described in Mitchell K, et al., 2003; Umbilical cord matrix stem cells (UCMSCs) described in Rachakatla R S, et al., 2007; Bovine umbilical cord-derived fibroblasts described in Saito S, et al., 2003; Wharton's Jelly-derived myofibroblast cells (WMFs) described in Schmidt D, et al., 2006; Mesenchymal cells in Wharton's jelly described in Wang H-S, et al., 2004; Umbilical cord matrix cells (UCM cells) described in Weiss M L, et al., 2003; Umbilical matrix stem cells (UCMS cells) described in Weiss M L, et al., 2006; Umbilical cord-derived stem cells (UCDS) described in Wu K H, et al., 2007.

Subamnion Derived Mesenchymal Stem Cells

Umbilical mesenchymal stem cells suitable for use in the methods and compositions described here may be derived from subamnion, including any of the following:

Umbilical mesenchymal stem cells described in Coppi P, et al., 2007; umbilical mesenchymal stem cells described in Chiavegato A, et al., 2007.

### Wharton's Jelly

The umbilical cord is enveloped in an epithelium and contains Wharton's jelly, in which two arteries and one vein are embedded.

“Wharton's Jelly,” also known as inter-laminar jelly, as the term is used in this document, refers to a mucous-connective tissue substance found in the umbilical cord. The components of Wharton's Jelly include a mucous connective tissue in which are found myfofibroblasts, fibroblasts, collagen fibers and an amorphous ground substance composed of hyaluronic acid and possibly other as yet uncharacterized cell populations. Wharton's jelly is one component of the umbilical cord matrix. Umbilical mesenchymal stem cells useful in the methods and compositions described in this document may be derived from Wharton's Jelly.

The epithelium is derived from the enveloping amnion. Wharton's jelly is a mucoid connective tissue comprising glycoprotein microfibrils coupled with collagen fibrils (Meyer, et al. Biochim Biophys Acta 1983; 755: 376-387). The interlaced collagen fibers and small, woven bundles are arranged to form a continuous soft skeleton that encases the umbilical vessels (Vizza et al Reprod Fertil Dev 1996; 8: 885-894). Mc Elreavey et al. Biochem Soc Trans 1991; 19: 29S described the isolation, culture and characterisation of fibroblast-like cells derived from the Wharton's jelly portion of human umbilical cord. Furthermore, Wharton's jelly also includes cells with the ultrastructural characteristics of myofibroblasts (Kobayashi et al. Early Hum Dev 1998; 51: 223-233).

### Derivation of Umbilical Mesenchymal Stem Cells

Umbilical mesenchymal stem cells suitable for use in the methods and compositions described here may be produced by any suitable means.

United States Patent Application No. US 2003/0161818 describes a method for obtaining stem cells from an umbilical cord matrix. The method comprises fractionating the umbilical cord matrix source of cells, the source substantially free of cord blood, into a fraction enriched with stem cells, and a fraction depleted of stem cells, and exposing the fraction enriched with stem cells to conditions suitable for cell proliferation. Methods of culture of umbilically derived mesenchymal stem cells are also described in this document.

Other documents such as U.S. Pat. No. 5,919,702 (Purchio), Mitchell et al. (Stem Cells 21:50-60, 2003), Romanov et al. (Stem Cells 21(1):105-110, 2003), U.S. Pat. No. 7,510,873 (Mistry) and U.S. Pat. Nos. 7,875,272 and 7,875,273 (Messina) also describe the derivation of mesenchymal stem cells from umbilical sources. The methods described there in may also be employed to produce umbilical mesenchymal stem cells suitable for use in the methods and compositions described in this document.

A general protocol for obtaining umbilical mesenchymal stem cells follows:

Umbilical mesenchymal stem cells as described in this document may be obtained in a number of ways. For example, they may be derived from umbilical tissue. The umbilical tissue may be processed, such as by dissection, mincing or washing, or any combination thereof.

The mesenchymal stem cell may be derived from such a tissue based on any suitable property, such as preferential adhesion. For example, the mesenchymal stem cell may be selected based on its ability to adhere to a substrate. The substrate may for example comprise plastic. Accordingly, the mesenchymal stem cell may be derived from umbilical tissue by allowing the mesenchymal stem cells in the umbilical tissue mass to adhere to plastic. For this purpose, the tissue may be placed on a vessel with one or more plastic surfaces. Such a vessel may comprise a culture vessel, for example a tissue culture plate. The vessel may be gelatinised. The mesenchymal stem cells may be allowed to migrate out of the tissue mass. They may be allowed to adhere to the surface of the vessel. The rest of the umbilical tissue may be removed, such as by washing off. Thus, the bulk of the tissue pieces may then be washed off, leaving a homogenous cell culture.

The tissue may be cultured in any suitable medium, such as Dulbecco's Modified Eagle Medium. The medium may be supplemented with any suitable supplement, such as serum replacement medium, EGF, FGF2, etc. The serum replacement medium, where present, may be included at any suitable concentration such as 10%. EGF, where present, may be included at any suitable concentration such as 5, 10, 15 or 20 ng/ml. FGF2, where present, may be included at any suitable concentration such as 5, 10, 15 or 20 ng/ml.

A specific protocol which may be used for deriving umbilical mesenchymal stem cells may comprise the following.

Collection of umbilical cords for this study was approved by the IRB of KK Women's and Children's Hospital. The cord was stored in DPBS+Gentamycin (10 ug/ml) s at 4° C. To isolate MSCs, the cord was cut into 3 cm long pieces and rinsed with DPBS+Gentamycin (10 ug/ml) to remove as much blood as possible. The cord was cut lengthwise and blood vessels were removed. The cord was digested with PBS containing 300 unit/ml collagenase, 1 mg/ml Hyaluronidase and 3 mM CaCl₂ for 1 hr at 37° C. water bath with occasional agitation. The cord pieces were crushed with forceps to release cells from the Wharton's jelly. The cord pieces were then digested with 0.05% Trypsin-EDTA for 30 min at 37° C. The cord pieces were again crushed with forceps to release cells from the Wharton's jelly. The cell suspensions were combined, washed and cultured as previously described²⁷.

The mesenchymal stem cell may be derived by optionally selecting a mesenchymal stem cell from other cells based on expression of a cell surface marker, as described in further detail below. The cell may therefore optionally be selected by detecting elevated expression of for example CD105 (Accession. Number NM_{—}000118.1) or CD73 (Accession Number NM_{—}002526.1), or both. The cell may be further optionally selected by detecting a reduced expression of CD24 (Accession Number NM_{—}013230.1). Thus, the mesenchymal stem cell may be obtained by selecting for cells which are CD105+ CD24−. The mesenchymal stem cell may be selected by labelling the cell with an antibody against the appropriate surface antigen and may be selected by fluorescence activated cell sorting (FACS) or magnetic cell sorting (MACS).

A further detailed example protocol for deriving umbilical mesenchymal stem cells, adapted from US 2003/0161818, follows:

Obtaining Umbilical Cord

In order to isolate umbilical mesenchymal stem cells, umbilical cord may be obtained under sterile conditions immediately following the termination of pregnancy (either full term or pre-term). The umbilical cord or a section thereof may be transported from the site of the delivery to a laboratory in a sterile container containing a preservative medium. One example of such a preservative medium may comprise Dulbecco's Modified Eagle's Medium (DMEM) with HEPES buffer.

The umbilical cord may be maintained and handled under sterile conditions prior to and during the collection of the stem cells from the matrix or Wharton's jelly. It may additionally be surface-sterilized by brief surface treatment of the cord with, for example, an aqueous (70% ethanol) solution or betadine, followed by a rinse with sterile, distilled water. The umbilical cord may be stored for up to about three hours at about 3-5 degrees C., but not frozen, prior to extraction of umbilical mesenchymal stem cells from the cellular source including the Wharton's Jelly umbilical component.

Wharton's jelly may be collected from the umbilical cord under sterile conditions by an appropriate method known in the art. For example, the cord may be cut transversely with a scalpel, for example, into approximately one inch sections, and each section may be transferred to a sterile container containing a sufficient volume of phosphate buffered saline (PBS) containing CaCl₂ (0.1 g/l) and MgCl₂.6H₂O (0.1 g/l) to allow surface blood to be removed from the section by gentle agitation. The section may then be removed to a sterile-surface where the outer layer of the section may be sliced open along the cord's longitudinal axis. The blood vessels of the umbilical cord (two veins and an artery) may be dissected away, for example, with sterile forceps and dissecting scissors, and the Wharton's jelly may be collected and placed in a sterile container, such as a 100 mm TC-treated Petri dish. The Wharton's jelly may then be cut into smaller sections, such as 2-3 mm³ for culturing.

Method of Obtaining Umbilical Mesenchymal Stem Cells from Wharton's Jelly

Wharton's jelly may be incubated in vitro in culture medium under appropriate conditions to permit the proliferation of any umbilical mesenchymal stem cells present therein. Any appropriate type of culture medium can be used to isolate the umbilical mesenchymal stem cells, such as, but not limited to DMEM. The culture medium may be supplemented with one or more components including, for example, fetal bovine serum, equine serum, human serum and one or more antibiotics and/or mycotics to control microbial contamination. Alternatively, serum free medium as known in the art may be used. Examples of antibiotics include but are not limited to penicillin G, streptomycin sulfate, amphotericin B, gentamycin, and nystatin, either alone or in combination.

Methods for the selection of the most appropriate culture medium, medium preparation, and cell culture techniques are well known in the art and are described in a variety of sources, including Doyle et al., (eds.), 1995, Cell and Tissue Culture: Laboratory Procedures, John Wiley & Sons, Chichester; and Ho and Wang (eds.), 1991, Animal Cell Bioreactors, Butterworth-Heinemann, Boston, which are incorporated herein by reference.

Another method relies on enzymatic dispersion of Wharton's Jelly with collagenase and isolation of cells by centrifugation followed by plating.

Establishment of Umbilical Mesenchymal Stem Cells in Cell Culture

The method involves fractionating the source of cells (Wharton's Jelly) into two fractions, one of which may be enriched with a stem cell and thereafter exposing the stem cells to conditions suitable for cell proliferation. The cell enriched isolate thus created comprises totipotent immortal stem cells.

After culturing Wharton's jelly for a sufficient period of time, for example, about 10-12 days, umbilical mesenchymal stem cells present in the explanted tissue will tend to have grown out from the tissue, either as a result of migration therefrom or cell division or both. These umbilical mesenchymal stem cells may then be removed to a separate culture vessel containing fresh medium of the same or a different type as that used initially, where the population of umbilical mesenchymal stem cells can be mitotically expanded.

Alternatively, the different cell types present in Wharton's jelly can be fractionated into subpopulations from which umbilical mesenchymal stem cells can be isolated. This may be accomplished using standard techniques for cell separation including, but not limited to, enzymatic treatment to dissociate Wharton's jelly into its component cells, followed by cloning and selection of specific cell types (for example, myofibroblasts, stem cells, etc.), using either morphological or biochemical markers, selective destruction of unwanted cells (negative selection), separation based upon differential cell agglutinability in the mixed population as, for example, with soybean agglutinin, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis, and fluorescence activated cell sorting (FACS). For a review of clonal selection and cell separation techniques, see Freshney, 1994, Culture of Animal Cells; A Manual of Basic Techniques, 3d Ed., Wiley-Liss, Inc., New York, which is incorporated herein by reference.

For example, Wharton's jelly may be cut into sections of approximately 2-3 mm.sup.3, and placed in a TC-treated Petri dish containing glass slides on the bottom of the Petri dish. The tissue sections may then be covered with another glass slide and cultured in a complete medium, such as for example RPMI 1640 containing 10% FRS 5% FS and antimicrobial compounds, including penicillin G (100 .mu.g/ml), streptomycin sulfate (100 .mu.g/ml), amphotericin (250.mu.g/ml), and gentamicin (10 .mu.g/ml), pH 7.4-7.6. The tissue may be preferably incubated at 37-39 degrees. C. and 5% CO.sub.2 for 10-12 days.

The medium may be changed as necessary by carefully aspirating the medium from the dish, for example, with a pipette, and replenishing with fresh medium. Incubation may be continued as above until a sufficient number or density of cells accumulates in the dish and on the surfaces of the slides. For example, the culture obtains approximately 70 percent confluence but not to the point of complete confluence. The original explanted tissue sections may be removed and the remaining cells may be trypsinized using standard techniques. After trypsinization, the cells may be collected, removed to fresh medium and incubated as above. The medium may be changed at least once at 24 hr post-trypsin to remove any floating cells. The cells remaining in culture may be considered to be umbilical mesenchymal stem cells.

Once the umbilical mesenchymal stem cells have been isolated, their population may be expanded mitotically. The stem cells should be transferred or “passaged” to fresh medium when they reach an appropriate density, such as 3×10⁴-cm² to 6.5×10⁴-cm², or, for example, when they reach a defined percentage of confluency on the surface of a culture dish. During incubation of the stem cells, cells can stick to the walls of the culture vessel where they can continue to proliferate and form a confluent monolayer. Alternatively, the liquid culture can be agitated, for example, on an orbital shaker, to prevent the cells from sticking to the vessel walls. The cells can also be grown on Teflon-coated culture bags.

### Characteristics of Umbilical Mesenchymal Stem Cells

The umbilical mesenchymal stem cells obtained by the methods and compositions described here may display one or more properties or characteristics of mesenchymal stem cells.

They may satisfy any one or more of the morphologic, phenotypic and functional criteria commonly used to identify mesenchymal stem cells⁹, as known in the art. The properties or characteristics may be as defined by The International Society for Cellular Therapy. In particular, they may display one or more characteristics as set out in Dominici et al (2006).

Morphology

The mesenchymal stem cells obtained by the methods and compositions described here may exhibit one or more morphological characteristics of mesenchymal stem cells.

The mesenchymal stem cells obtained by the methods described here may display a typical fingerprint whorl at confluency.

The mesenchymal stem cells obtained may form an adherent monolayer with a fibroblastic phenotype. The mesenchymal stem cell may be capable of adhering to plastic.

They may have an average population doubling time of between 72 to 96 hours. The optimal culture may be at 25% to 85% confluency or 15-50,000 cells per cm².

Antigen Profile

Furthermore, the mesenchymal stem cells obtained may display a surface antigen profile which is similar or identical to mesenchymal stem cells.

The mesenchymal stem cells obtained by the methods described here may lack or display reduced expression of one or more pluripotency marker, such as of Oct-4, SSEA-4 and TRa1-60, for example at the polypeptide level. They may display transcript expression of one or both of OCT4 and SOX2, but at reduced levels compared to embryonic stem cells such as hES3 human ESCs. The levels of expression may be 2 times lower, 5 times lower or 10 times lower or more compared to embryonic stem cells.

The obtained mesenchymal stem cells may display a “typical” MSC-like surface antigen profile. They may for example show expression of one or more markers associated with mesenchymal stem cells. These may include expression of any one or more of the following: CD29, CD44, CD49a, CD49e, CD105, CD166, MHC I. The umbilical mesenchymal stem cells may for example show reduced or absent expression of any one or more markers whose absence of expression is associated with mesenchymal stem cells. Thus, the umbilical mesenchymal stem cells may display reduced or lack of expression of any one or more of HLA-DR, CD34 and CD45. The umbilical mesenchymal stem cells may in particular comprise CD29+, CD44+, CD49a+ CD49e+, CD105+, CD166+, MHC I+, CD34⁻ and CD45⁻ cells.

The transformed mesenchymal stem cell, cell culture or cell line may display a ZFP42. The transformed mesenchymal stem cell, cell culture or cell line may display a reduced expression of one or more, such as all, of OCT4, NANOG and SOX2. The transformed mesenchymal stem cell, cell culture or cell line may display no detectable alkaline phosphatase activity.

Maintenance in Culture

The transformed mesenchymal stem cell, cell culture or cell line may be maintainable in cell culture for greater than 10, 20, 30, 40 or more generations.

The transformed mesenchymal stem cell, cell culture or cell line may have a substantially stable karyotype or chromosome number when maintained in cell culture for at least 10 generations. The transformed mesenchymal stem cell, cell culture or cell line may be such that it does not substantially induce formation of teratoma when transplanted to a recipient animal. The recipient animal may comprise an immune compromised recipient animal. The time period may be after 3 weeks, such as after 2 to 9 months. The transformed mesenchymal stem cell, cell culture or cell line may be such that it not teratogenic when implanted in SCID mice.

The transformed mesenchymal stem cell, cell culture or cell line may be such that it is negative for mouse-specific c-mos repeat sequences and positive for human specific alu repeat sequences.

Differentiation Potential

The mesenchymal stem cells obtained may be differentiated into any mesenchymal lineage, using methods known in the art and described below. Thus, the mesenchymal stem cells obtained by the methods and compositions described here may display a differentiation potential that include adipogenesis, chondrogenesis and osteogenesis⁹. The transformed mesenchymal stem cell, cell culture or cell line may be capable of undergoing osteogenesis, adipogenesis or chondrogenesis, such as capable of differentiating into osteocytes, adipocytes or chondrocytes.

Gene Expression Profile

The transformed mesenchymal stem cell, cell culture or cell line may have a substantially stable gene expression pattern from generation to generation.

The transformed mesenchymal stem cell, cell culture or cell line may be such that any two or more, such as all, transformed mesenchymal stem cells obtainable by the method exhibit substantially identical gene expression profiles. It may be such that the gene expression correlation coefficient between any two or more isolates of transformed mesenchymal stem cells obtained by the method is greater than 0.9. It may be such that any two or more, such as all, isolates of transformed mesenchymal stem cells obtainable by the method are substantially similar or identical (such as homogenous) with each other. It may be such that the gene expression correlation coefficient between a transformed mesenchymal stem cell obtainable by the method and cells of a parental culture is greater than 0.8.

Proliferative Capacity

The mesenchymal stem cells obtained as described, e.g., hESC-MSCs, can have a substantial proliferative capacity in vitro. In some embodiments, the mesenchymal stem cells obtained may undergo at least 10 population doublings while maintaining a normal diploid karyotype. The mesenchymal stem cells may be capable of undergoing at least 20-30 population doublings while maintaining a normal diploid karyotype. In some embodiments, the mesenchymal stem cells display a stable gene expression and surface antigen profile throughout this time.

The mesenchymal stem cells obtained may be such that they do not display any defects, such as chromosomal aberrations and/or alterations in gene expression. In some embodiments, such defects are not evident until after 10 passages, such as after 13 passages, for example after 15 passages.

Homogeneity

The mesenchymal stem cells obtained may display a high degree of uniformity. In other words, the mesenchymal stem cells obtained from different umbilical sources may display one or more, such as a plurality, of uniform or distinct characteristics that are shared with each other. They may display one or more, such as a plurality, of uniform or distinct characteristics that are shared with other mesenchymal stem cells, such as a human embryonic stem cell derived mesenchymal stem cells (hESC-MSCs), as for example described in WO2007/027157 or WO2007/027156.

For example, the mesenchymal stem cells may be such that any two or more, such as all, mesenchymal stem cells selected by the method exhibit substantially identical gene expression profiles. The gene expression correlation coefficient between any two or more isolates of mesenchymal stem cells obtained by the method may be greater than 0.8, such as greater than 0.85 or 0.9. The gene expression correlation coefficient between any two or more different passages, such as successive passages, of mesenchymal stem cells obtained by the method may be greater than 0.8, such as greater than 0.85 or 0.9. The gene expression correlation coefficient between a mesenchymal stem cell obtained by the method and hESC-MSCs, such as for example described in WO2007/027157 or WO2007/027156, may be greater than 0.8, such as greater than 0.85 or 0.9.

Any two or more, such as all, isolates of mesenchymal stem cells obtained by the method may be substantially similar or identical (such as homogenous) with each other.

Telomerase Activity

The mesenchymal stem cells so derived may comprise telomerase activity. The telomerase activity may be elevated or up-regulated compared to a control cell such as a cell which is not a mesenchymal stem cell. For example, the control cell may comprise a differentiated cell, such as a differentiated cell in the mesenchymal lineage, for example, an osteocyte, adipocyte or chondrocyte.

Telomerase activity may be determined by means known in the art, for example, using TRAP activity assay (Kim et al., Science 266:2011, 1997), using a commercially available kit (TRAPeze® XK Telomerase Detection Kit, Cat. s7707; Intergen Co., Purchase N.Y.; or Te1oTAGGG™ Telomerase PCR ELISA plus, Cat. 2,013,89; Roche Diagnostics, Indianapolis). hTERT expression can also be evaluated at the mRNA level by RT-PCR. The LightCycler TeIoTAGGG™ hTERT quantification kit (Cat. 3,012,344; Roche Diagnostics) is available commercially for research purposes.

For example, relative telomerase activity may be measured by real time quantitative telomeric repeat amplification protocol. This qPCR-based assay quantifies product generated in vitro by telomerase activity present in the samples. The relative telomerase activity which is directly proportional to the amount of telomerase products may be assessed by the threshold cycle number (or Ct value) for one pg protein cell lysate. Hues9.E1 refers to a previously described hESC-MSCs line and HEK is a human embryonic kidney cell line. The Ct value for each umbilical MSCs may be taken as a mean for multiple passages, for example, 2, 3, 4 etc passages. The passages may for example comprise three passages, such as P16, P18, and P20. For the control cells, such as Hues9.E1 the mean may be for two passages, such as P20 and P22. The assay may be performed multiple times, such as in triplicate, for each passage.

A specific example of a telomerase detection method is provided in the Examples.

The mesenchymal stem cells derived by the methods described here may have a Ct value, as assayed by such a method, of 25 or more, such as 26 or more or 27 or more. The Ct value may be for example 28 or more, 29 or more, 30 or more, 31 or more or 32 or more.

Cardioprotective Ability

The mesenchymal stem cell so derived or a medium conditioned by such a cell may comprise cardioprotective ability, as described in the Examples. The cardioprotection may comprise restoration or maintenance of cardiac function during ischemia and/or reperfusion.

Cardioprotection may be assayed in any suitable model system, such as a mouse model of acute myocardial infarction (AMI). In such an assay, AMI is induced in mice by permanent ligation of the left anterior descending coronary artery as described in Salto-Tellez M, Yung Lim S, El-Oakley R M, Tang T P, ZA A L, et al. (2004) Myocardial infarction in the C57BL/6J mouse: a quantifiable and highly reproducible experimental model. Cardiovasc Pathol 13: 91-97.

100 μl of 10× X concentrated conditioned medium or non-conditioned medium (control) made as described above is then administered to the mice via an osmotic pump placed at the jugular vein over the next 72 hours. Heart function in these mice is assessed by MRI three weeks later.

Cardioprotection may also be assayed in a pig/mouse model of myocardial ischemia/reperfusion (MI/R) injury (Timmers L, Lim S-K, Arslan F, et al. Reduction of myocardial infarct size by human mesenchymal stem cell conditioned medium. Stem Cell Research. 2008;1:129-137). In this assay, injury is induced by a temporary occlusion of the left circumflex artery in pig or the LAD in mouse followed by removal of occlusion to initiate reperfusion.

The cardioprotection assays described above may also be used to test for cardioprotection especially in chronic ischemia. This and the other MI/R injury model essentially evaluate cardioprotection in different clinical indications.

The mesenchymal stem cell so derived or a medium conditioned by such a cell may be capable of alleviating reperfusion injury. This may be assayed using a porcine model of ischemia-reperfusion as described in WO2008/020815.

A brief protocol for assaying cardioprotective ability of umbilical mesenchymal stem cell conditioned medium follows. MI may be induced by 30 minutes occlusion of left coronary artery (LCA) by ligating the artery with a suture. Subsequent reperfusion may be initiated by releasing the suture. Five minutes before reperfusion, mice may be intravenously infused with 200 μl saline diluted conditioned medium containing 3 μg protein for Hues9.E1 (hESC-MSC) CM or 150 μg protein for umbilical MSC CM via the tail vein. Control animals may be infused with 200 μl saline. After 24 hours reperfusion, infarct size (IS) as a percentage of the area at risk (AAR) may be assessed using Evans' blue dye injection and TTC staining as described previously in reference 26.

Briefly, just before excision of the heart for analysis, the LCA may be re-ligated as in the induction of ischemia, Evans blue dye may be infused into the aorta and the AAR may be defined by the area not stained by Evans' blue dye. The heart may then be excised and cross sections of the heart may be stained with TTC. Viable myocardium is stained red by TTC while non-viable myocardium is not stained. Relative infarct size may be measured as the area of non-viable myocardium not stained by TTC relative to the AAR risk defined by the area not stained by Evans' blue dye.

Infarct Size

The mesenchymal stem cell so derived or a medium conditioned by such a cell may have the ability to reduce infarct size. The infarct size may be reduced by 10% or more, 20% or more, 30% or more, 40% or more, 50% or more, 60% or more or 70% or more compared with an animal that is treated with a non-conditioned medium or saline.

Assay for Infarct Size

Infarct size may for example be assayed using the following method.

Just prior to excision of the heart, the LCxCA (pigs) or LCA (mice) is religated at exactly the same spot as for the induction of the MI. Evans blue dye is infused through the coronary system to delineate the area at risk (AAR).

The heart is then excised, the LV is isolated and cut into 5 slices from apex to base. The slices are incubated in 1% triphenyltetrazolium chloride (TTC, Sigma-Aldrich Chemicals, Zwijndrecht, the Netherlands) in 37° C. Sorensen buffer (13.6 g/L KH₂PO₄+17.8 g/L Na₂H PO₄.2H₂O, pH 7.4) for 15 minutes to discriminate infarct tissue from viable myocardium.

All slices are scanned from both sides, and in each slide, the infarct area is compared with area at risk and the total area by use of digital planimetry software (Image After correction for the weight of the slices, infarct size is calculated as a percentage of the AAR and of the LV.

Oxidative Stress

The mesenchymal stem cells produced by the method described here may be capable of reducing oxidative stress.

The oxidative stress may be reduced by 10% or more, 20% or more, 25% or more, 30% or more, 40% or more, 50% or more, 60% or more or 70% or more compared with an animal that is treated with a non-conditioned medium or saline.

The reduction of oxidative stress may be assayed in an in vitro assay of hydrogen peroxide (H₂O₂)-induced cell death.

Assay for Oxidative Stress

The reduction of oxidative stress may for example be assayed using an in vitro assay of hydrogen peroxide (H₂O₂)-induced cell death. In summary, hydrogen peroxide (H₂O₂)-mediated oxidative stress is induced in human leukemic CEM cells and cell viability is monitored by Trypan blue-exclusion. Human leukemic CEM cells are incubated with conditioned medium or mesenchymal stem cell (with saline as a control) and treated with 50 μM H₂O₂ to induce oxidative stress. Cell viability is assessed using Trypan Blue exclusion at 12, 24, 36 and 48 hours after H₂O₂ treatment.

The reduction of oxidative stress may further be assayed using an in vivo assay of DNA oxidation. In vivo oxidative stress may also be assayed as follows. Pigs are treated with the particle, conditioned medium or mesenchymal stem cell (with saline as a control). Tissue sections of pig heart are obtained. Nuclear oxidative stress in tissue sections of treated and untreated pigs is quantified by 8-OHdG immunostaining for oxidized DNA. The tissue sections are assayed for intense nuclear staining indicative of DNA oxidation and oxidative stress.

Homogeneity

The mesenchymal stem cells produced by the method described here may be similar or identical (such as homogenous) in nature. That is to say, mesenchymal stem cell (MSC) clones isolated by the protocol may show a high degree of similarity or identity with each other, whether phenotypically or otherwise.

Similarity or identity may be gauged by a number of ways and measured by one or more characteristics. For example, the clones may be similar or identical in gene expression. The method may be such that any two or more mesenchymal stem cells selected by the method exhibit substantially identical or similar gene expression profiles, that is to say, a combination of the identity of genes expressed and the level to which they are expressed. For example, substantially all of the mesenchymal stem cells isolated may exhibit substantially identical or similar gene expression profiles.

Homogeneity of gene expression may be measured by a number of methods. Genome-wide gene profiling may be conducted using, for example, array hybridisation of extracted RNA as described in the Examples. Total RNA may be extracted and converted into cDNA, which is hybridised to an array chip comprising a plurality of gene sequences from a relevant genome. The array may comprise NCBI Reference Sequence (RefSeq) genes, which are well characterised genes validated, annotated and curated on an ongoing basis by National Center for Biotechnology Information (NCBI) staff and collaborators.

Gene expression between samples may then be compared using analysis software. In one embodiment, the similarity or identity of gene expression may be expressed as a “correlation coefficient”. In such measures, a high correlation coefficient between two samples indicates a high degree of similarity between the pattern of gene expression in the two samples. Conversely, a low correlation coefficient between two samples indicates a low degree of similarity between the pattern of gene expression in the two samples. Normalisation may be conducted to remove systematic variations or bias (including intensity bias, spatial bias, plate bias and background bias) prior to data analysis.

Correlation tests are known in the art and include a T-test and Pearson's test, as StatSoft, Tulsa, Okla., ISBN: 1884233597 (also StatSoft, Inc. (2006). Electronic Statistics Textbook. Tulsa, Okla.: StatSoft. WEB: http://www.statsoft.com/textbook/stathome.html). Reference is made to Khojasteh et al., 2005, A stepwise framework for the normalization of array CGH data, BMC Bioinformatics 2005, 6:274. An Intra-class correlation coefficient (ICC) may also be conducted, as described in Khojasteh et al, supra.

For example, a Pearson's test may be conducted to generate a Pearson's correlation coefficient. A correlation coefficient of 1.0 indicates an identical gene expression pattern.

For such purposes, the cDNA may be hybridised to a Sentrix HumanRef-8 Expression BeadChip and scanned using a Illumina BeadStation 500×. The data may be extracted, normalised and analysed using Illumina BeadStudio (Illumina, Inc, San Diego, Calif., USA). It will be clear to the reader however that any suitable chip and scanning hardware and software (which outputs a correlation measurement) may be used to assay similarity of gene expression profile.

The gene expression correlation coefficient between any two isolates as for example measured by the above means may be 0.65 or more. The gene expression correlation coefficient may be 0.70 or more, such as 0.80 or more, such as 0.85 or more, such as 0.90 or more. The coefficient may be 0.91 or more, 0.92 or more, 0.93 or more, 0.94 or more, 0.95 or more, 0.96 or more, 0.97 or more, 0.98 or more, 0.99 or more or 1.0.

In some embodiments, the method described here generates mesenchymal stem cells whose gene expression correlation coefficient between any two or more isolates of mesenchymal stem cells so obtained is in the same order as, or slightly less than, the correlation coefficient between technical replicates of the same RNA sample, performed a period of time apart such as 1 month apart. For example, the gene expression correlation coefficient between any two or more isolates of mesenchymal stem cells may be 0.70 or more, such as 0.80 or more, such as 0.85 or more, such as 0.90 or more. The coefficient may be 0.91 or more, 0.92 or more, 0.93 or more, 0.94 or more, 0.95 or more, 0.96 or more, 0.97 or more, 0.98 or more, 0.99 or more or 1.0.

The gene expression correlation coefficients may be in such ranges for cells which have undergone the derivation, selection or sorting procedure described above. The gene expression correlation coefficient between the majority of isolates, such as all isolates, may be

[symbol]

Thus, as shown in the Examples, the correlation coefficient shows a high degree of similarity between the five mesenchymal stem cell cultures obtained. The correlation value of the gene expression profile between different passages of each culture is greater than 0.9. The correlation value of the gene expression profile between the five cultures is greater than 0.9.

Accordingly, we provide for a method of generating mesenchymal stem cells which are substantially similar or identical (such as homogenous) with each other. The isolates may display a near identical gene expression profile.

As well as the “internal” homogeneity described above (i.e., homogeneity between the isolates of mesenchymal stem cells from the method), homogeneity may also be assessed between such isolates and other cells or cell types. In particular, comparisons may be made with mesenchymal stem cells derived by other methods, such as a human ESC-MSC culture (for example as described in WO2007/027157 or WO2007/027156). In some embodiments, therefore, the mesenchymal stem cells obtained by the methods and compositions described here display a gene expression profile which is similar to, homogenous with, or identical with such a human ESC-MSC culture. Thus, the mesenchymal stem cells obtained may show a correlation coefficient of gene expression of greater than 0.5, such as greater than 0.6, such as greater than 0.7, such as greater than 0.8, such as greater than 0.9, as with such a human ESC-MSC culture (for example as described in WO2007/027157 or WO2007/027156).

### Transformed Umbilical Mesenchymal Stem Cells

Transformed umbilical mesenchymal stem cells may be obtained or derived using the methods and compositions described here from an umbilical mesenchymal stem cell, such as by genetic engineering. The genetic engineering may comprise transformation with a suitable transforming agent, such as an oncogene.

As shown in the Examples, transformed umbilical mesenchymal stem cells are capable of producing exosomes for which the ratio of AV+ exosomes (exosomes capable of binding to annexin V) to CTB+ exosomes (exosomes capable of binding to cholera toxin B chain) is higher, for example 10 to 15× higher, than those secreted by transformed embryonic stem cell derived mesenchymal stem cells.

We therefore describe methods for immortalizing or transforming such cell lines by transfecting a umbilical mesenchymal stem cell with a vector comprising at least one

[symbol]

Examples of transforming genes include: (1) nuclear oncogenes such as v-myc, N-myc, T antigen and Ewing's sarcoma oncogene (Fredericksen et al. (1988) Neuron 1:439-448; Bartlett, P. et al. (1988) Proc. Natl. Acad. Sci. USA 85:3255-3259, and Snyder, E. Y. et al. (1992) Cell 68:33-51), (2) cytoplasmic oncogenes such as bcr-abl and neurofibromin (Solomon, E. et al. (1991) Science 254:1153-1160), (3) membrane oncogenes such as neu and ret (Aaronson, A. S. A. (1991) Science 254:1153-1161), (4) tumor suppressor genes such as mutant p53 and mutant Rb (retinoblastoma) (Weinberg, R. A. (1991) Science 254:1138-1146), and (5) other transforming genes such as Notch dominant negative (Coffman, C. R. et al. (1993) Cell 23:659-671). Examples of oncogenes include v-myc and the SV40 T antigen.

Transforming genes may further include telomerase gene (Tert) as well as oncogenes such as Akt, ras, EGF receptor etc.

Foreign (heterologous) nucleic acid such as encoding transforming genes may be introduced or transfected into mesenchymal stem cell. A mesenchymal stem cell which harbors foreign DNA is said to be a genetically-engineered cell. The foreign DNA may be introduced using a variety of techniques. In one embodiment, foreign DNA, including DNA encoding a transforming gene, is introduced into mesenchymal stem cell using the technique of retroviral transfection. Recombinant retroviruses harboring the gene(s) of interest are used to introduce marker genes, such as the E. coli β-galactosidase (lacZ) gene, or oncogenes. The recombinant retroviruses are produced in packaging cell lines to produce culture supernatants having a high titer of virus particles (generally 10⁵ to 10⁶ pfu/ml). The recombinant viral particles are used to infect cultures of the mesenchymal stem cell by incubating the cell cultures with medium containing the viral particles and 8 μg/ml polybrene for three hours. Following retroviral infection, the cells are rinsed and cultured in standard medium. The infected cells are then analyzed for the uptake and expression of the foreign DNA. The cells may be subjected to selective conditions which select for cells that have taken up and expressed a selectable marker gene.

In another example, the foreign DNA may be introduced using the technique of calcium-phosphate-mediated transfection. A calcium-phosphate precipitate containing DNA encoding the gene(s) of interest, such a transforming gene, is prepared using the technique of Wigler et al. (1979) Proc. Natl. Acad. Sci. USA 76:1373-1376. Cultures of the mesenchymal stem cell are established in tissue culture dishes. Twenty four hours after plating the cells, the [symbol] added. The cells are incubated at room temperature for 20 minutes. Tissue culture medium containing 30 μM chloroquine is added and the cells are incubated overnight at 37° C. Following transfection, the cells are analyzed for the uptake and expression of the foreign DNA. The cells may be subjected to selection conditions which select for cells that have taken up and expressed a selectable marker gene.

As used herein, the term ‘non-transformed cells’ means cells which are able to grow in vitro without the need to immortalize the cells by introduction of a virus or portions of a viral genome containing an oncogene(s) which confers altered growth properties upon cells by virtue of the expression of viral genes within the transformed cells. These viral genes typically have been introduced into cells by means of viral infection or by means of transfection with DNA vectors containing isolated viral genes.

As used herein, the term ‘genetically-engineered cell’ refers to a cell into which a foreign (i.e., non-naturally occurring) nucleic acid, e.g., DNA, has been introduced. The foreign nucleic acid may be introduced by a variety of techniques, including, but not limited to, calcium-phosphate-mediated transfection, DEAE-mediated transfection, microinjection, retroviral transformation, protoplast fusion and lipofection. The genetically-engineered cell may express the foreign nucleic acid in either a transient or long-term manner. In general, transient expression occurs when foreign DNA does not stably integrate into the chromosomal DNA of the transfected cell. In contrast, long-term expression of foreign DNA occurs when the foreign DNA has been stably integrated into the chromosomal DNA of the transfected cell.

As used herein, an ‘immortalized cell’ or a ‘transformed cell’ means a cell which is capable of growing indefinitely in culture due to the introduction of an ‘immortalizing gene’ or ‘transforming gene’ which confers altered growth properties upon the cell by virtue of expression of the immortalizing or transforming gene(s) within the genetically engineered cell. Immortalizing genes and transforming genes can be introduced into cells by means of viral infection or by means of transfection with vectors containing isolated viral nucleic acid encoding one or more oncogenes. Viruses or viral oncogenes are selected which allow for the immortalization but preferably not the transformation of cells. Immortalized cells may be such that they grow indefinitely in culture but do not cause tumours when introduced into animals.

As used herein, the term ‘transformed cell’ refers to a cell having a transforming gene introduced into it and having the ability to grow indefinitely in culture. ‘Transformation’ refers to the generation of a transformed cell.

### Umbilical Mesenchymal Stem Cell Cultures and Cell Lines

It will be evident that such an umbilical mesenchymal stem cell that is obtained by the methods described here, including a transformed umbilical mesenchymal stem cell, may be maintained as a cell or developed into a cell culture or a cell line.

Accordingly, in this document, and where appropriate, the term “umbilical mesenchymal stem cell” should be taken also to include reference to a corresponding cell culture, i.e., an umbilical mesenchymal stem cell culture or a corresponding cell line, i.e., an umbilical mesenchymal stem cell line. In general, the umbilical mesenchymal stem cell, cell culture or cell line may be maintained in culture in the same or similar conditions and culture media as described above for derivation.

The umbilical mesenchymal stem cell culture or cell line, including transformed umbilical mesenchymal stem cell culture or cell line, may be cultured under hypoxic conditions.

### Umbilical Mesenchymal Stem Cell Conditioned Medium

We further provide a medium which is conditioned by culture of the umbilical mesenchymal stem cells, including transformed umbilical mesenchymal stem cells. The umbilical mesenchymal stem cell or transformed umbilical mesenchymal stem cell may be cultured under hypoxic conditions.

Such a conditioned medium is referred to in this document as a ‘umbilical mesenchymal stem cell conditioned medium’ and is described in further detail below.

Such a conditioned medium may comprise molecules secreted, excreted, released, or otherwise produced by the umbilical mesenchymal stem cells. The conditioned medium may comprise one or more molecules of the mesenchymal stem cells, for example, polypeptides, nucleic acids, carbohydrates or other complex or simple molecules. The conditioned medium may also comprise one or more activities of the mesenchymal stem cells. The conditioned medium may be used in place of, in addition to, or to supplement the mesenchymal stem cells themselves. Thus, any purpose for which mesenchymal stem cells are suitable for use in, conditioned media may similarly be used for that purpose.

Such a conditioned medium, and combinations of any of the molecules comprised therein, including in particular proteins or polypeptides, may be used to supplement the activity of, or in place of, the umbilical mesenchymal stem cells, for the purpose of for example treating or preventing a disease. Thus, where it is stated that the mesenchymal stem cells obtained by the methods described here may be used for a particular purpose, it will be evident that media conditioned by such mesenchymal stem cells may be equally used for that purpose.

Conditioned medium may be made by any suitable method. For example, it may be made by culturing mesenchymal stem cells in a medium, such as a cell culture medium, for a predetermined length of time. Any number of methods of preparing conditioned medium may be employed, including the “Conditioned Medium Preparation Protocol” set out below.

The mesenchymal stem cells may in particular comprise those produced by any of the methods described in this document. The conditioned medium will comprise polypeptides secreted by the mesenchymal stem cells, as described in the Examples.

A particular example of a protocol for producing conditioned medium, which is not intended to be limiting, is as follows.

An example protocol for preparing conditioned medium from umbilical mesenchymal stem cells may comprise a “Conditioned Medium Preparation Protocol”, which is as follows:

### Umbilical Mesenchymal Stem Cell Conditioned Medium Preparation Protocol

The secretion may be prepared as set out in the Examples by growing the umbilical MSCs in a chemically defined serum free culture medium for three days. 80% confluent cultures may be washed three times with PBS, and then cultured overnight in a chemically defined medium consisting of DMEM media without phenol red supplemented with 1× Insulin-Transferrin-Selenoprotein, 10 ng/ml Recombinant Human FGF2, 10 ng/ml Recombinant Human EGF, 1×glutamine-penicillin-streptomycin, and 55 μM β-mercaptoethanol overnight. The cultures may then be washed three times with PBS and fresh chemically defined medium may then be added. All medium components may be obtained from Invitrogen. The cultures may be maintained in this medium for three days. This conditioned medium (CM) may then be collected, clarified by centrifugation, concentrated 50 times using tangential flow filtration with MW cut-off of 100 kDa (Satorius, Goettingen, Germany) and sterilized by filtration through a 220 nm filter.

The cells may be cultured under hypoxic conditions.

The conditioned medium may be used in therapy as is, or after one or more treatment steps. For example, the conditioned medium may be UV treated, filter sterilised, etc. One or more purification steps may be employed. In particular, the conditioned media may be concentrated, for example by dialysis or ultrafiltration. For example, the medium may be concentrated using membrane ultrafiltration with a nominal molecular weight limit (NMWL) of for example 3K.

For the purposes described in this document, for example, treatment or prevention of disease, a dosage of conditioned medium comprising 15-750 mg protein/100 kg body weight may be administered to a patient in need of such treatment.

### Umbilical Mesenchymal Stem Cell Particle

We describe a particle which is derivable from a umbilical mesenchymal stem cell (MSC). Such a particle is referred to in this document as a “umbilical mesenchymal stem cell particle”.

The umbilical mesenchymal stem cell particle may be derivable from the umbilical MSC by any of several means, for example by secretion, budding or dispersal from the umbilical MSC, such as under hypoxic conditions. For example, the umbilical mesenchymal stem cell particle may be produced, exuded, emitted or shed from the umbilical MSC for example under hypoxic conditions. Where the umbilical MSC is in cell culture, the umbilical mesenchymal stem cell particle may be secreted into the cell culture medium.

The umbilical mesenchymal stem cell particle may in particular comprise a vesicle. The umbilical mesenchymal stem cell particle may comprise an exosome. The umbilical mesenchymal stem cell particle described here may comprise any one or more of the properties of the exosomes described herein.

The umbilical mesenchymal stem cell particle may comprise vesicles or a flattened sphere limited by a lipid bilayer. The umbilical mesenchymal stem cell particle may comprise inward budding of the endosomal membrane. The umbilical mesenchymal stem cell particle may have a density of ˜1.13-1.19 g/ml and may float on sucrose gradients. The umbilical mesenchymal stem cell particle may be enriched in cholesterol and sphingomyelin, and lipid raft markers such as GM1, GM3, flotillin and the src protein kinase Lyn. The umbilical mesenchymal stem cell particle may comprise one or more proteins present in umbilical mesenchymal stem cells or umbilical mesenchymal stem cell conditioned medium (MSC-CM), such as a protein characteristic or specific to the umbilical MSC or umbilical MSC-CM. They may comprise RNA, for example miRNA.

We provide a umbilical mesenchymal stem cell particle which comprises one or more genes or gene products found in umbilical MSCs or medium which is conditioned by culture of umbilical. MSCs. The umbilical mesenchymal stem cell particle may comprise molecules secreted by the umbilical MSC. Such a umbilical mesenchymal stem cell particle, and combinations of any of the molecules comprised therein, including in particular proteins or polypeptides, may be used to supplement the activity of, or in place of, the umbilical MSCs or medium conditioned by the umbilical MSCs for the purpose of for example treating or preventing a disease.

The umbilical mesenchymal stem cell particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9⁻, CD63, CD81 and CD82. In particular, the umbilical mesenchymal stem cell particle may comprise one or more tetraspanins. The umbilical mesenchymal stem cell particles may comprise mRNA and/or microRNA.

The term “particle” as used in this document may be taken to mean a discrete entity.

The particle may be something that is isolatable from a umbilical mesenchymal stem cell (umbilical MSC) or umbilical mesenchymal stem cell conditioned medium (umbilical MSC-CM). The umbilical mesenchymal stem cell particle may be responsible for at least an activity of the umbilical MSC or umbilical MSC-CM. The umbilical mesenchymal stem cell particle may be responsible for, and carry out, substantially most or all of the functions of the umbilical MSC or umbilical MSC-CM. For example, the umbilical mesenchymal stem cell particle may be a substitute (or biological substitute) for the umbilical MSC or umbilical MSC-CM.

The umbilical mesenchymal stem cell particle may be used for any of the therapeutic purposes that the umbilical MSC or umbilical MSC-CM may be put to use.

The umbilical mesenchymal stem cell particle preferably has at least one property of a umbilical mesenchymal stem cell. The umbilical mesenchymal stem cell particle may have a biological property, such as a biological activity. The umbilical mesenchymal stem cell particle may have any of the biological activities of an umbilical MSC. The umbilical mesenchymal stem cell particle may for example have a therapeutic or restorative activity of an umbilical MSC.

The Examples show that media conditioned by umbilical MSCs (such as umbilical mesenchymal stem cell conditioned media or umbilical MSC-CM, as described below) comprise biological activities of umbilical MSC and are capable of substituting for the umbilical MSCs themselves. The biological property or biological activity of an umbilical MSC may therefore correspond to a biological property or activity of a umbilical mesenchymal stem cell conditioned medium. Accordingly, the umbilical mesenchymal stem cell particle may comprise one or more biological properties or activities of a umbilical mesenchymal stem cell conditioned medium (umbilical MSC-CM).

The conditioned cell culture medium such as a Umbilical Mesenchymal Stem Cell Conditioned Medium (umbilical MSC-CM) may be obtained by culturing a umbilical mesenchymal stem cell (umbilical MSC), a descendent thereof or a cell line derived therefrom in a cell culture medium; and isolating the cell culture medium. The umbilical mesenchymal stem cell may be produced by a process comprising obtaining a cell by dispersing a embryonic stem (ES) cell colony. The cell, or a descendent thereof, may be propagated in the absence of co-culture in a serum free medium comprising FGF2. Further details are provided elsewhere in this document.

### Isolation of Umbilical Mesenchymal Stem Cell Particle

The umbilical MSC particle may be produced or isolated in a number of ways. Such a method may comprise isolating the particle from a umbilical mesenchymal stem cell (umbilical MSC). Such a method may comprise isolating the umbilical MSC particle from a

[symbol]

The umbilical MSC particle may be isolated for example by being separated from non-associated components based on any property of the umbilical MSC particle. For example, the umbilical MSC particle may be isolated based on molecular weight, size, shape, composition or biological activity.

The conditioned medium may be filtered or concentrated or both during, prior to or subsequent to separation. For example, it may be filtered through a membrane, for example one with a size or molecular weight cut-off It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the umbilical MSC particle may be used to track its activity during fractionation of the umbilical mesenchymal stem cell conditioned medium (umbilical MSC-CM). As an example, light scattering, refractive index, dynamic light scattering or UV-visible detectors may be used to follow the umbilical MSC particles. For example, a therapeutic activity such as cardioprotective activity may be used to track the activity during fractionation.

The following paragraphs provide a specific example of how a umbilical MSC mesenchymal stem cell particle such as an exosome may be obtained.

A umbilical mesenchymal stem cell particle may be produced by culturing mesenchymal stem cells in a medium to condition it. The umbilical mesenchymal stem cells may comprise HuES9.E1 cells. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof.

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more.

The conditioned medium may be subject to liquid chromatography such as HPLC. The conditioned medium may be separated by size exclusion. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector.

Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rₕ of particles in this peak is about 55-65 nm. Such fractions comprise umbilical MSC mesenchymal stem cell particles such as exosomes.

With regard to transformed umbilical mesenchymal stem cells, we demonstrate that these are capable of producing exosomes for which the ratio of AV+ exosomes (exosomes capable of binding to annexin V) to CTB+ exosomes (exosomes capable of binding to cholera toxin B chain) is higher, for example 10 to 15× higher, than those secreted by transformed embryonic stem cell derived mesenchymal stem cells. Accordingly, this feature may be used to isolate exosomes produced by umbilical mesenchymal stem cells, for example, by exposing a umbilical mesenchymal stem cell conditioned medium to immobilised Annexin V such as in a column.

### Delivery of Umbilical MSC Particles

The umbilical MSC particles as described in this document may be delivered to the human or animal body by any suitable means.

We therefore describe a delivery system for delivering a umbilical MSC particle as described in this document to a target cell, tissue, organ, animal body or human body, and methods for using the delivery system to deliver umbilical MSC particles to a target.

The delivery system may comprise a source of umbilical MSC particles, such as a container containing the umbilical MSC particles. The delivery system may comprise a dispenser for dispensing the umbilical MSC particles to a target.

Accordingly, we provide a delivery system for delivering a umbilical MSC particle, comprising a source of umbilical MSC particles as described in this document together with a dispenser operable to deliver the umbilical. MSC particles to a target.

We further provide for the use of such a delivery system in a method of delivering umbilical MSC particles to a target.

Delivery systems for delivering fluid into the body are known in the art, and include injection, surgical drips, catheters (including perfusion catheters) such as those described in U.S. Pat. No. 6,139,524, for example, drug delivery catheters such as those described in U.S. Pat. No. 7,122,019.

Delivery to the lungs or nasal passages, including intranasal delivery, may be achieved using for example a nasal spray, puffer, inhaler, etc as known in the art (for example as shown in U.S. Design Pat. No. D544,957.

Delivery to the kidneys may be achieved using an intra-aortic renal delivery catheter, such as that described in U.S. Pat. No. 7,241,273.

It will be evident that the particular delivery should be configurable to deliver the required amount of umbilical MSC particles at the appropriate interval, in order to achieve optimal treatment.

The umbilical MSC particles may for example be used for the treatment or prevention of atherosclerosis. Here, perfusion of umbilical MSC particles may be done intravenously to stabilize atherosclerotic plaques or reduce inflammation in the plaques. The umbilical MSC particles may be used for the treatment or prevention of septic shock by intravenous perfusion.

The umbilical MSC particles may be used for the treatment or prevention of heart umbilical MSC particles to retard remodeling or retard heart failure. The umbilical MSC particles may be used for the treatment or prevention of lung inflammation by intranasal delivery.

The umbilical MSC particles may be used for the treatment or prevention of dermatological conditions e.g. psoriasis. Long term delivery of umbilical MSC particles may be employed using transdermal microinjection needles until the condition is resolved.

It will be evident that the delivery method will depend on the particular organ to which the umbilical MSC particles is to be delivered, and the skilled person will be able to determine which means to employ accordingly.

As an example, in the treatment of cardiac inflammation, the umbilical MSC particles may be delivered for example to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen.

Any variety of coronary catheter, or a perfusion catheter, may be used to administer the umbilical MSC particles. Alternatively the umbilical MSC particles may be coated or impregnated on a stent that is placed in a coronary vessel.

### Diseases Treatable

The proteome of mesenchymal stem cells obtained by the methods described here may be analysed.

The mesenchymal stem cells thus obtained my have an expression profile which is similar to that of mesenchymal stem cells, such as hESC-MSCs described in WO2007/027157 or WO2007/027156. Thus, mesenchymal stem cells derived by our methods may have significant biological similarities to their counterparts, e.g., in their ability to secrete paracrine factors. Accordingly, the mesenchymal stem cells described here may be used for any purpose for which hESC-MSCs such as those described in WO2007/027157 or WO2007/027156 are suitable.

The umbilical mesenchymal stem cells, conditioned medium or particles such as [symbol] and tissue differentiation including vascularization, hematopoiesis and skeletal development, or whose repair or treatment involves any one or more of these biological processes. Similarly, the proteins expressed, such as secreted, by the umbilical mesenchymal stem cells, singly or in combination, such as in the form of a conditioned medium, may be used to supplement the activity of, or in place of, the umbilical mesenchymal stem cells, for the purpose of for example treating or preventing such diseases.

The umbilical mesenchymal stem cells, conditioned medium or particles such as exosomes derived therefrom may be used to activate signaling pathways in cardiovascular biology, bone development and hematopoiesis such as Jak-STAT, MAPK, Toll-like receptor, TGF-beta signaling and mTOR signaling pathways. Thus, the umbilical mesenchymal stem cells, proteins expressed by them, etc, may be used to prevent or treat a disease in which any of these signaling pathways is involved, or whose aetiology involves one or more defects in any one or more of these signaling pathways.

Accordingly, umbilical mesenchymal stem cells, conditioned medium or particles such as exosomes derived therefrom may be used to treat cardiac failure, bone marrow disease, skin disease, burns and degenerative diseases such as diabetes, Alzheimer's disease, Parkinson's disease and cancer.

Umbilical mesenchymal stem cells, conditioned medium or particles such as exosomes derived therefrom may also be used to treat myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

They may be used to aid wound healing, scar reduction, bone formation, a bone graft or bone marrow transplantation in an individual.

We provide for the use of umbilical mesenchymal stem cells, conditioned medium or particles such as exosomes derived therefrom in the regulation of pathways including any one or more of the following: cytoskeletal regulation by Rho GTPase, cell cycle, integrin FGF signaling pathway, EGF receptor signaling pathway, angiogenesis, plasminogen activating cascade, blood coagulation, glycolysis, ubiquitin proteasome pathway, de novo purine biosynthesis, TCA cycle, phenylalanine biosynthesis, heme biosynthesis.

We provide also for the use of umbilical mesenchymal stem cells, conditioned medium or particles such as exosomes derived therefrom in the regulation of processes including any one or more of the following: cell structure and motility, cell structure, cell communication, cell motility, cell adhesion, endocytosis, mitosis, exocytosis, cytokinesis, cell cycle, immunity and defense, cytokine/chemokine mediated immunity, macrophage-mediated immunity, granulocyte-mediated immunity, ligand-mediated signaling, cytokine and chemokine mediated signaling pathway, signal transduction, extracellular matrix protein-mediated signaling, growth factor homeostasis, receptor protein tyrosine kinase signaling pathway, cell adhesion-mediated signaling, cell surface receptor mediated signal transduction, JAK-STAT cascade, antioxidation and free radical removal, homeostasis, stress response, blood clotting, developmental processes, mesoderm development, skeletal development, angiogenesis, muscle development, muscle contraction, protein metabolism and modification, proteolysis, protein folding, protein complex assembly, amino acid activation, intracellular protein traffic, other protein targeting and localization, amino acid metabolism, protein biosynthesis, protein disulfide-isomerase reaction, carbohydrate metabolism, glycolysis, pentose-phosphate shunt, other polysaccharide metabolism, purine metabolism, regulation of phosphate metabolism, vitamin metabolism, amino acid biosynthesis, pre-mRNA processing, translational regulation, mRNA splicing.

We further provide for the use of umbilical mesenchymal stem cells, conditioned medium or particles such as exosomes derived therefrom in the supply of functions including any one or more of the following: signaling molecule, chemokine, growth factor, cytokine, interleukin, other cytokine, extracellular matrix, extracellular matrix structural protein, other extracellular matrix, extracellular matrix glycoprotein, protease, metalloprotease, other proteases, protease inhibitor, metalloprotease inhibitor, serine protease inhibitor, oxidoreductase, dehydrogenase, peroxidase, chaperone, chaperonin, Hsp 70 family chaperone, other chaperones, synthetase, synthase and synthetase, select calcium binding protein, aminoacyl-tRNA synthetase, lyase, isomerase, other isomerase, ATP synthase, hydratase, transaminase, other lyase, other enzyme regulator, select regulatory molecule, actin binding cytoskeletal protein, cytoskeletal protein, non-motor actin binding protein, actin and actin related protein, annexin, tubulin, cell adhesion molecule, actin binding motor protein, intermediate filament, ribonucleoprotein, ribosomal protein, translation factor, other RNA-binding protein, histone, calmodulin related protein, vesicle coat protein.

Furthermore, the umbilical mesenchymal stem cells, conditioned medium or particles such as exosomes derived therefrom may be used to treat diseases which these functions may have a role in, or whose repair or treatment involves any one or more of these biological processes. Similarly, the proteins expressed by the mesenchymal stem cells, singly or in combination, such as in the form of a conditioned medium, may be used to supplement the activity of, or in place of, the mesenchymal stem cells, for the purpose of for example treating or preventing such diseases.

The gene products expressed by the mesenchymal stem cells obtained herein may be used to activate important signaling pathways in cardiovascular biology, bone development and hematopoiesis such as Jak-STAT, MAPK, Toll-like receptor, TGF-beta signaling and mTOR signaling pathways. Accordingly, the umbilical mesenchymal stem cells, conditioned medium or particles such as exosomes derived therefrom may be used to prevent or treat a disease in which any of these signaling pathways is involved, or whose aetiology involves one or more defects in any one or more of these signaling pathways.

Accordingly, umbilical mesenchymal stem cells, conditioned medium or particles such as exosomes derived therefrom may be used to treat cardiac failure, bone marrow disease, skin disease, burns and degenerative diseases such as diabetes, Alzheimer's disease, Parkinson's disease and cancer.

Such umbilical mesenchymal stem cells, conditioned medium or particles such as exosomes derived therefrom may also be used to treat myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

The umbilical mesenchymal stem cells, conditioned medium or particles such as exosomes derived therefrom may be used to aid wound healing, scar reduction, bone formation, a bone graft or bone marrow transplantation in an individual.

In particular, umbilical mesenchymal stem cells, conditioned medium or particles such as exosomes derived therefrom may be used to regulate the processes involved in vascularisation, hematology (specifically immune processes) or musculoskeletal development, etc.

Such a composition may be used for any purpose the conditioned medium may be used. Unless the context dictates otherwise, the term “conditioned medium” should be taken to include not only cell culture medium exposed to umbilical MSCs as well as such a composition comprising one or more, such as substantially all, the polypeptides which are present in the conditioned medium.

Furthermore, any one or more proteins secreted from the mesenchymal stem cells described here, including in the form of conditioned media, may be used for the same purposes as the hESC-MSCs as described in WO2007/027157 or WO2007/027156.

The umbilical mesenchymal stem cells may also be used as sources for any of the proteins secreted or expressed by them. We therefore provide for a method of producing a polypeptide comprising obtaining a mesenchymal stem cell as described, culturing the mesenchymal stem cell and isolating the polypeptide from the mesenchymal stem cell, such as from a medium in which the mesenchymal stem cell is growing.

Dermatologic Disorders

Umbilical mesenchymal stem cells, conditioned medium or particles such as exosomes derived therefrom obtained by the methods described in this document may be used for the treatment of dermatologic disorders.

Therefore the topical application of such umbilical mesenchymal stem cells, conditioned medium or particles such as exosomes derived therefrom on cutaneous wounds or dermatological lesions or disorders such as dermatitis or psoriasis improves healing and reduces scarring. It should also maintain homeostasis. The umbilical mesenchymal stem cells, conditioned medium or particles such as exosomes derived therefrom may be delivered in liposome-based emulsion, gel or cream formulations and part of standard wound dressing. The umbilical mesenchymal stem cells, conditioned medium or particles such as exosomes derived therefrom may also be used as a supplement in cosmetic skincare product to promote skin repair and healing.

A suitable animal model to test the efficacy of such umbilical mesenchymal stem cells, conditioned medium or particles such as exosomes derived therefrom on cutaneous disorders is a mouse model of dermatitis. Epicutaneous sensitization of mice is performed as described earlier [A72, A73]. Briefly, 50 ng of Blo t 5 in 100 μl of PBS or PBS alone are applied to 1 cm² gauze and patched to the skin with a transparent dressing (Smith Nephew). This procedure is repeated twice over a period of 50 days. CM and NCM are then applied to 1 cm2 gauze and patched to the skin as described above for varying period of time.

For histological examination of skin inflammation, specimens are obtained from the patched skins and fixed in 10% buffered neutral formalin immediately.

Asthma and Allergy

Umbilical mesenchymal stem cells, conditioned medium or particles such as exosomes derived therefrom obtained by the methods described in this document may be used for the treatment of asthma and allergy.

Asthma is a complex disease with an equally complex etiology caused by a poorly characterized set of genetic and environmental factors. The resulting pathology is immune dysregulation leading to chronic inflammation of the airways and subepithelial fibrosis characterized by increase in smooth muscle mass and increased deposition of extracellular matrix proteins and subsequently, reduced lung function.

Current therapies include modulating several factors or signaling pathways e.g. the ECM, integrins, and mesenchymal cell function, toll-like receptors, growth factors such as TGF-β and EGF and the IL6 pathway.

To investigate the effects of such umbilical mesenchymal stem cells, conditioned medium or particles such as exosomes derived therefrom on chronic airway inflammation and airway, epicutaneous sensitization of mice are performed. After 50 days, the patched mice are anesthetized and receive intranasal challenge with 50 μg of Blo t 5 for three consecutive days. Twenty-four hours after the last dose, airway hyperresponsivensess (AHR) is measured using invasive BUXCO. The mice are anesthetized and given umbilical mesenchymal stem cells, conditioned medium or particles such as exosomes derived therefrom intranasally for three consecutive days. Twenty-four hours after the last dose, airway hyperresponsivensess (AHR) is measured. BAL fluid is collected after another twenty-four hours. Following bronchoalveolar lavage collection, lungs are fixed with 10% neutralized formalin.

To investigate the effects of umbilical mesenchymal stem cells, conditioned medium or particles such as exosomes derived therefrom on acute airway inflammation and airway:

A Blot t 5 specific Th2 cell line which secretes high level of IL-4, IL-5, IL-13 and with undetectable level of IFN-γ, is used to establish a mouse allergy model. Briefly, sensitization of naïve mice is done by adoptive transfer of 2.5×10⁶ Blo t 5 specific Th2 cells intravenously in each mouse. These mice are anesthetized and intranasal (IN) challenged with 50 μg of Blo t 5 for three consecutive days. Twenty-four hours after the last IN challenge, airway hyperresponsivensess (AHR) is measured using invasive BUXCO. The mice are then anesthetized and given umbilical mesenchymal stem cells, conditioned medium or particles such as exosomes derived therefrom intranasally for three consecutive days. BAL fluid is collected at forty-eight hours after the last Blo t 5 challenge. Following bronchoalveolar lavage collection, lungs are fixed with 10% neutralized formalin for histopathological analysis.

In clinical practice, the use of umbilical mesenchymal stem cells, conditioned medium or particles such as exosomes derived therefrom to treat lung disease may be administered effectively using standard aerosol therapy.

Other Diseases

Umbilical mesenchymal stem cells, conditioned medium or particles such as exosomes derived therefrom obtained by the methods described in this document may be used for the treatment of other diseases.

In general, we disclose that umbilical mesenchymal stem cells, conditioned medium or particles such as exosomes derived therefrom are useful in restoring homeostasis and promoting tissue repair in pathological conditions where the initial injury induced inflammation and immune dysregulation leads to chronic tissue remodeling that includes fibrosis and loss of function. Other diseases include renal ischemic injury, cystic fibrosis, sinusitis and rhinitis.

Orthopedics

Umbilical mesenchymal stem cells, conditioned medium or particles such as exosomes derived therefrom obtained by the methods described in this document may be used for the treatment of orthopedic disorders.

Current therapeutic strategies for repair of musculoskeletal tissue often include the use of a biomaterial (ceramics or polymers) not only to provide mechanical support but also as a scaffold to promote cell migration, cell adhesion, proliferation and differentiation to initiate vascularization and ultimately new bone formation. Based on the computation analysis of such conditioned medium, incorporation of umbilical mesenchymal stem cells, conditioned medium or particles such as exosomes derived therefrom into the scaffold design may enhance cell migration, proliferation, adhesion, skeletal differentiation and vascularization of the scaffold.

To test the effect of umbilical mesenchymal stem cells, conditioned medium or particles such as exosomes derived therefrom on bone regeneration in defects that would otherwise have led to atrophic nonunions, New Zealand white rabbits receive a 15-mm critical size defect on one radius, which is filled with a suitable matrix such as a collagen sponge or hydrogel coated with either CM or NCM. Radiographs are obtained every 3 weeks. After 6 or 12 weeks, animals are killed. New bone is measured by microCT scans and vascularity is measured using anti-CD31 staining of endothelial cells in the implant. There should be increased vascularity at the least initially and also increased new bone formation.

To test the effects of umbilical mesenchymal stem cells, conditioned medium or particles such as exosomes derived therefrom on cartilage repair, a rabbit model of osteochondral injury is used. Umbilical mesenchymal stem cells, conditioned medium or particles such as exosomes derived therefrom are coated on a suitable scaffold such as collagen or hydrogel and implanted into 3-mm osteochondral knee defects. For clinical applications, umbilical mesenchymal stem cells, conditioned medium or particles such as exosomes derived therefrom may be used by incorporating the umbilical mesenchymal stem cells, conditioned medium or particles such as exosomes derived therefrom into existing scaffolds or bone grafts.

Bone Marrow Transplantation

MSCs have been shown to enhance bone marrow transplantation and ameliorate graft versus host disease. Transplantation of MSCs has been shown to improve the outcome of allergenic transplantation by promoting hematopoietic engraftment and limiting GVHD. It is postulated that MSCs mediate these effects through the enhancement of the hematopoietic stem cell niche and the induction of tolerance and reduce GVHD, rejection of allergenic tissue transplant and modulation of inflammation, possibly through the secretion of soluble factors.

Potential clinical applications of such conditioned medium by MSCs: expansion of hematopoietic stem cell population in vitro by supplementing culture media with CM or in vivo by infusing umbilical mesenchymal stem cells, conditioned medium or particles such as exosomes derived therefrom with hematopoietic stem cells during transplantation, ameliorate GVHD by intravenous infusion of umbilical mesenchymal stem cells, conditioned medium or particles such as exosomes derived therefrom or induction of immune tolerance to transplanted cells or tissues by intravenous infusion of umbilical mesenchymal stem cells, conditioned medium or particles such as exosomes derived therefrom as part of the pre and post-transplant therapy.

Heart Disease

The umbilical mesenchymal stem cells, conditioned medium or particles such as exosomes derived therefrom described here may be used for treatment or prevention of heart disease.

Heart disease is an umbrella term for a variety for different diseases affecting the heart. As of 2007, it is the leading cause of death in the United States, England, Canada and Wales, killing one person every 34 seconds in the United States alone. Heart disease includes any of the following.

Coronary Heart Disease

Coronary artery disease is a disease of the artery caused by the accumulation of atheromatous plaques within the walls of the arteries that supply the myocardium. Angina pectoris (chest pain) and myocardial infarction (heart attack) are symptoms of and conditions caused by coronary heart disease. Over 459,000 Americans die of coronary heart disease every year. In the United Kingdom, 101,000 deaths annually are due to coronary heart disease.

Cardiomyopathy

Cardiomyopathy is the deterioration of the function of the myocardium (i.e., the actual heart muscle) for any reason. People with cardiomyopathy are often at risk of arrhythmia and/or sudden cardiac death. Extrinsic cardiomyopathies—cardiomyopathies where the primary pathology is outside the myocardium itself comprise the majority of cardiomyopathies. By far the most common cause of a cardiomyopathy is ischemia.

The World Health Organization includes as specific cardiomyopathies: Alcoholic cardiomyopathy, coronary artery disease, congenital heart disease, nutritional diseases affecting the heart, ischemic (or ischaemic) cardiomyopathy, hypertensive cardiomyopathy, valvular cardiomyopathy, inflammatory cardiomyopathy.

Also included are:

Cardiomyopathy secondary to a systemic metabolic disease

Intrinsic cardiomyopathies (weakness in the muscle of the heart that is not due to an identifiable external cause)

Dilated cardiomyopathy (DCM, the most common form, and one of the leading indications for heart transplantation. In DCM the heart (especially the left ventricle) is enlarged and the pumping function is diminished)

Hypertrophic cardiomyopathy (HCM or HOCM, a genetic disorder caused by various mutations in genes encoding sarcomeric proteins. In HCM the heart muscle is thickened, which can obstruct blood flow and prevent the heart from functioning properly),

Arrhythmogenic right ventricular cardiomyopathy (ARVC, which arises from an electrical disturbance of the heart in which heart muscle is replaced by fibrous scar tissue. The right ventricle is generally most affected)

Restrictive cardiomyopathy (RCM, which is the least common cardiomyopathy. The walls of the ventricles are stiff, but may not be thickened, and resist the normal filling of the heart with blood).

Noncompaction Cardiomyopathy—the left ventricle wall has failed to properly grow from birth and such has a spongy appearance when viewed during an echocardiogram.

Cardiovascular Disease

Cardiovascular disease is any of a number of specific diseases that affect the heart itself and/or the blood vessel system, especially the veins and arteries leading to and from the heart. Research on disease dimorphism suggests that women who suffer with cardiovascular disease usually suffer from forms that affect the blood vessels while men usually suffer from forms that affect the heart muscle itself. Known or associated causes of cardiovascular disease include diabetes mellitus, hypertension, hyperhomocysteinemia and hypercholesterolemia.

Types of cardiovascular disease include atherosclerosis

Ischaemic Heart Disease

Ischaemic heart disease is disease of the heart itself, characterized by reduced blood supply to the organs. This occurs when the arteries that supply the oxygen and the nutrients gets stopped and the heart will not get enough of the oxygen and the nutrients and will eventually stop beating.

Heart Failure

Heart failure, also called congestive heart failure (or CHF), and congestive cardiac failure (CCF), is a condition that can result from any structural or functional cardiac disorder that impairs the ability of the heart to fill with or pump a sufficient amount of blood throughout the body. Cor pulmonale is a failure of the right side of the heart.

Hypertensive Heart Disease

Hypertensive heart disease is heart disease caused by high blood pressure, especially localised high blood pressure. Conditions that can be caused by hypertensive heart disease include: left ventricular hypertrophy, coronary heart disease, (Congestive) heart failure, hypertensive cardiomyopathy, cardiac arrhythmias, inflammatory heart disease, etc.

Inflammatory heart disease involves inflammation of the heart muscle and/or the tissue surrounding it. Endocarditis comprises inflammation of the inner layer of the heart, the endocardium. The most common structures involved are the heart valves. Inflammatory cardiomegaly. Myocarditis comprises inflammation of the myocardium, the muscular part of the heart.

Valvular Heart Disease

Valvular heart disease is disease process that affects one or more valves of the heart. The valves in the right side of the heart are the tricuspid valve and the pulmonic valve. The valves in the left side of the heart are the mitral valve and the aortic valve. Included are aortic valve stenosis, mitral valve prolapse and valvular cardiomyopathy.

[The above text is adapted from Heart disease. (2009 Feb. 3). In Wikipedia, The Free Encyclopedia. Retrieved 06:33, Feb. 20, 2009, from http://en.wikipedia.org/w/index.php?title=Heart disease&oldid=268290924]

### EXAMPLES

### Example 1

### Materials and Methods—Derivation of Cord MSCs

Collection of umbilical cords for this study was approved by the IRB of KK Women's and Children's Hospital. The cord was stored in DPBS+Gentamycin (10 ug/ml) s at 4° C. To isolate MSCs, the cord was cut into 3 cm long pieces and rinsed with DPBS+Gentamycin (10 ug/ml) to remove as much blood as possible. The cord was cut lengthwise and blood vessels were removed. The cord was digested with PBS containing 300 unit/ml collagenase, 1 mg/ml. Hyaluronidase and 3 mM CaCl₂ for 1 hr at 37° C. water bath with occasional agitation. The cord pieces were crushed with forceps to release cells from the Wharton's jelly. The cord pieces were then digested with 0.05% Trypsin-EDTA for 30 min at 37° C. The cord pieces were again crushed with forceps to release cells from the Wharton's jelly. The cell suspensions were combined, washed and cultured as previously described²⁷.

### Example 2

### Materials and Methods—Oncogenic Transformation of Cord MSC

Myc transformation of cord MSCs was performed as previously described³⁰. Briefly, the cord MSCs were infected with a lentivirus carrying the c-myc gene. The c-myc cDNA was amplified from pMXs-hc-MYC³² using primers PTDmyc (5′ GAA TTC GAA TGC CCC TCA ACG TTA GC 3′) and PTDmyca (5′ CTC GAG CGC ACA AGA GTT CCG TAG C 3′). The amplified fragment was inserted into pDrive vector and sequenced. The c-myc fragment was digested and cloned as a Xhol(filled)/EcoRl fragment into a Smal/EcoRl digested pLVX-puro vector with compatible ends (Clontech, www.clontech.com). Lentiviral particles were produced using Lenti-X HT Packaging System (Clontech, www.clontech.com) according to the manufacturer's protocol. Viral titer was determined using by a Lenti-X™ qRT-PCR titration kit (Clontech, www.clontech.com). The MSCs²⁴ were plated at 10⁶ cells per 10 cm dish and infected with viruses at a MOI=5 in the presence of 4 μg/ml polybrene overnight. The next day, culture medium was replaced with fresh medium. Selection began 48 hours after infection by replacing the culture medium with one containing puromycin (2 μg/ml). After three days of selection, cells were expanded as per human ESC-derived huES9.E1 MSCs. Clonal lines from each of the three independently infected cell cultures were derived by limiting dilution. When the cloned cells were expanded to 10⁷ cells (or a confluent 15 cm culture dish), the cells were designated p1. Three clonal lines were generated and named E1-myc 16.1, E1-myc 16.2 and E1-myc 16.3 lines, respectively. Integration of the c-myc or GFP transgene was confirmed by amplifying genomic DNA using specific primers for exon2 and exon3 of c-myc respectively: 5′-GCCCCTGGTGCTCCATGAGGAGACACC′-3′ and 5′-ACATTCTCCTCGGTGTCCGAGG-3′ using the following PCR conditions: one cycle of 94, 2 min; 32 cycles of 94° C., 15 s; 60° C., 30 s; 72° C., 90 s and one cycle of 72° C., 5 min. The PCR products were resolved on a 1% agarose gel.

Differentiation of the myc-MSCs to adipocytes, chondrocytes and osteocytes was performed using adipogenic, chondrogenic and osteogenic hMSC Differentiation BulletKits, respectively (Lonza, Walkersville, Md.) as per manufacturer' instructions. Karyotyping by G-banding was performed by the Cytogenetics Laboratory, KKH.

### Example 3

### Materials and Methods—Telomerase Activity

Relative telomerase activity was measured by SYBR® Green real time quantitative telomeric repeat amplification protocol assay using a modified method as described by Wege H. et al³³. Briefly, 3 million cells were harvested and cell lysate was prepared using a commercially available mammalian cell extraction kit (Cat K269-500-1, BioVision, www.biovision.com). The composition of the reagents for the PCR amplification was 1 μg of protein cell lysate, 10 μl of 2× SYBR Green Super Mix (Cat 170-8880, BioRad, Singapore) with 0.1 μg of TS primer (5′-AATCCGTCGAGCAGACTT-3′), 0.1 μg of ACX primer (5′-GCGCGG[CTTACC]3CTAACC-3′) and 10 mM EGTA in a total volume of 25 μl. The reaction was first incubated at 25° C. for 20 min to allow the telomerase in the cell lysate to elongate the TS primers followed by 2 min incubation at 95° C. to inactivate telomerase activity and denature the primers. The telomerase product was amplified by PCR for 40 cycles of 95° C., 30 s; 60° C., 90 s. The relative telomerase activity was assessed against that of HEK293

[symbol]

### Example 4

### Materials and Methods—Rate of Cell Cycling

To assess cell cycle rate, 2×10⁷ cells were pre-labeled with 2 ml of 10 μM CFDA (Molecular Probe, Eugene, Oreg.) in PBS at 37° C. for 15 minutes, cultured for 24 hours and then replated at 5×104 cells per well in 6-well coated with gelatin. At 0, 24, 48, and 72 hours, cells from duplicate wells were harvested, and fixed in 2% paraformaldehyde, and analyzed on a FACSpIus (Becton Dickinson; San Jose, Calif.). The number of cell cycles per 24 hours was calculated assuming that each halving of cellular fluorescence represented one cell division. Therefore, the number of cell cycles per 24 hours (n) was calculated as n=lg(F−Fn)/1g2 where F is initial average cellular fluorescence and Fn is the average cellular fluorescence after 24 hours. The number of cell cycles was then plotted against time to derive the average time per cell cycle.

### Example 5

### Materials and Methods—Surface Antigen Analysis

Expression of cell surface antigens on HuES9.E1 and CMSC3A1 MSCs was analyzed using flow cytometry as previously described³⁰. The cells were tryspinized for 5 minutes, centrifuged, resuspended in culture media and incubated in a bacterial culture dish for 1 hour in a 37° C., 5% CO₂ incubator. The cells were collected, centrifuged, washed in 2% FBS. 2.5×10⁵ cells were then incubated with each of the following conjugated monoclonal antibodies: CD29-PE, CD44-FITC, CD49a-PE, CD49e-PE, CD105-FITC, CD166-PE, CD73-FITC, CD34-FITC, CD45-FITC, HLADR-PE, and MHC1-PE (PharMingen, San Diego, Calif.) for 60 min on ice. After incubation, cells were washed and resuspended in 2% FBS. Nonspecific fluorescence was determined by incubation of similar cell aliquots with isotype-matched mouse monoclonal antibodies (PharMingen, San Diego, Calif.). Data were analyzed by collecting 20,000 events on a BD FACSCalibur™ Flow Cytometer (BD Biosciences, San Jose, Calif.) instrument using CELLQuest software.

### Example 6

### Materials and Methods—Quantitation of myc RNA Transcript by qRT-PCR

20 ηg cellular RNA was converted to cDNA using a High-Capacity cDNA Reverse Transcription Kit (Life Technologies, CA, USA). The cDNA was then amplified by one cycle of 94, 10 min; 40 cycles of 94° C., 15 s; 60° C., 60 s and one cycle of 95° C., 15 s, 60° C. 60 s, 95° C., 15 s with primer sets specific for either myc or actin transcript on the StepOnePlus Real-Time PCR system (Applied Biosystems, www.appliedbiosystems.com.sg). The myc-specific primer set is 5′-CCCGCCCCTGTCCCCTAGCCG-3′ and 5′-AGAAGGGTGTGACCGCAACGTAG3′.

### Example 7

### Materials and Methods—Illumina Gene Chip Analysis

Total RNA was prepared in technical triplicates from different passages of MSCs using Illumina® TotalPrep RNA Amplification Kit (Ambion, Inc., Austin, Tex.). The MScs were HuES9-E1 MSCs at p15 and p16; E1-myc 21.1 at p3, p4, and p5; E1-myc 16.3 line at p4, p7, and p8; CMSC3A1 at p4, p5, and p6; CMSC3A3 at p4, p5, and p6; and cord MSCs at p1 and p2. 500 ηg RNA was converted to biotinylated cRNA using the Illumina RNA Amplification Kit (Ambion, Inc., Austin, Tex.) according to the manufacturer's directions. 750 ng of the biotinylated cRNA were hybridized to the Sentrix HumanRef-8 Expression BeadChip Version 3 (Illumina, Inc., San Diego, Calif.). Washing and scanning were performed according to the Illumina BeadStation 500× manual. The data were analyzed using Genespring GX 10. Quantile normalization was performed by a shift to 75^{th} percentile, and the normalized data were baseline transformed to the median of all samples.

### Example 8

### Materials and Methods—HPLC Purification of Microparticles

The instrument setup consisted of a liquid chromatography system with a binary pump, an auto injector, a thermostated column oven and a UV-visible detector operated by the Class VP software from Shimadzu Corporation (Kyoto, Japan). The Chromatography columns used were TSK Guard column SWXL, 6×40 mm and TSK gel G4000 SWXL, 7.8×300 mm from Tosoh Corporation (Tokyo, Japan). The following detectors, Dawn 8 (light scattering), Optilab (refractive index) and QELS (dynamic light scattering) were connected in series following the UV-visible detector. The last three detectors were from Wyatt Technology Corporation (California, USA) and were operated by the ASTRA software. The components of the sample were separated by size exclusion i.e. the larger molecules will elute before the smaller molecules. The eluent buffer was 20 mM phosphate buffer with 150 mM of NaCl at pH 7.2. This buffer was filtered through a pore size of 0.1 μm and degassed for 15 minutes before use. The chromatography system was equilibrated at a flow rate of 0.5 ml/min until the signal in Dawn 8 stabilized at around 0.3 detector voltage units. The UV-visible detector was set at 220 ηm and the column was oven equilibrated to 25° C. The elution mode was isocratic and the run time was 40 minutes. The volume of sample injected ranged from 50 to 100 μl. The hydrodynamic radius, Rh was computed by the QELS and Dawn 8 detectors. The highest count rate (Hz) at the peak apex was taken as the Rh. Peaks of the separated components visualized at 220 ηm were collected as fractions for further characterization studies.

### Example 9

### Materials and Methods—Low Oxygen Culture

5×10⁶ umbilical cord-derived MSCs immortalized with the cMyc gene were seeded in a 15-cm culture dish (˜60% confluent) and allowed to attach overnight. They were then cultured under normoxia (20.9% O₂) or hypoxia (1% O₂) at 5% CO₂ and 95% humidity at 37° C. for 24 h. Each of the culture media was then replaced with fresh medium equilibrated to either normoxic or hypoxic conditions, and this medium was conditioned for 72 h. All hypoxia-related work was performed in the Xvivo integrated system of closed incubator and closed hood with a controlled level of oxygen (Biospherix, NY, USA). The relative level of exosomes in the conditioned medium was determined by extracting exosomes and measuring CD9 protein level in the extract. Exosomes were extracted by cholera toxin B chain affinity chromatography and CD9 in the extracts was measured by ELISA using CD9 specific antibody.

### Example 10

### Materials and Methods—Testing Secretion for Cardioprotection

The secretion was prepared by growing the transformed MSCs in a chemically defined serum free culture medium for three days as previously described³⁴. Briefly, cells at p12 were first expanded in serum-containing culture medium as described above. At p15, 80% confluent cell cultures were washed three times with PBS and then incubated in a chemically defined medium consisting of DMEM without phenol red (Invitrogen Corporation, Carlsbad, Calif.) and supplemented with insulin, transferrin, and selenoprotein (ITS) (Invitrogen Corporation, Carlsbad, Calif.), 5 ng/ml FGF2 (Invitrogen Corporation, Carlsbad, Calif.), 5 ng/ml PDGF AB (Peprotech, Rocky Hill, N.J.), glutamine-penicillin-streptomycin, and β-mercaptoethanol overnight. The cell culture was then washed with PBS and replaced with fresh chemically defined medium for another three days to produce the conditioned medium. This CM was collected and clarified by centrifugation at 500×g. The clarified CM concentrated 50 times by reducing its volume by a factor of 50 using a tangential flow filtration system with membrane MW cut-off of 100 kDa (Satorius, Goettingen, Germany). The use of a membrane MW cut-off of 100 kDa allow molecules with MW of less 100 kDa to pass through the filter resulting a preferential loss of molecules less than 100 kDa. The concentrated CM was then sterilized by filtration through a 220 nm filter.

The CM was tested in a mouse model of ischemia and reperfusion injury. MI was induced by 30 minutes left coronary artery (LCA) occlusion and subsequent reperfusion. Five minutes before reperfusion, mice were intravenously infused with 200 μl saline solution of 0.3 μg exosome protein purified from culture medium conditioned by myc-MSCs. Control animals were infused with 200 μl saline. After 24 hours reperfusion, infarct size (IS) as a percentage of the area at risk (AAR) was assessed using Evans' blue dye injection and TTC staining as described previously²⁷.

### Example 11

### Materials and Methods—Statistical Analysis

Two-way ANOVA with post-hoc Dunnett was used to test the difference in infarct size between groups. Correlation coefficient of each pairs of array was assessed using Pearson correlation test.

### Example 12

### Results—Karyotype

To generate independently cloned lines, cord MSC cultures at p3 were independently infected and placed under puromycin drug selection. The surviving cells were re-plated at low density ranging from 20,000-50,000 cells per 10 cm plate to produce colonies that were physically well separated. Colonies were picked and transferred into 96-well plates. Three colonies were eventually selected to establish cell lines, CMSC3A1, CMSC3A2 and CMSC3A3. Each of the cell lines was karyotyped and one of the clones, CMSC3A1 has a normal 46 XY.

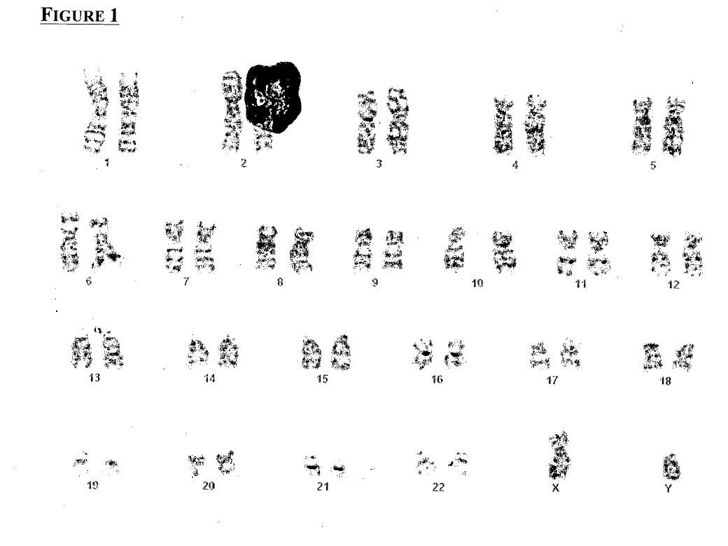

The results are shown in FIG. 1.

### Example 13

### Results—Myc Transcript Level

The level of myc transcripts in CMSC3A1 was higher than that in the huES9.E1 MSCs which were MSCs derived from hESCs²⁴, but lower than E1-myc 16.3 MSCs, the myc-transformed huES9.E1 MSCs³⁰

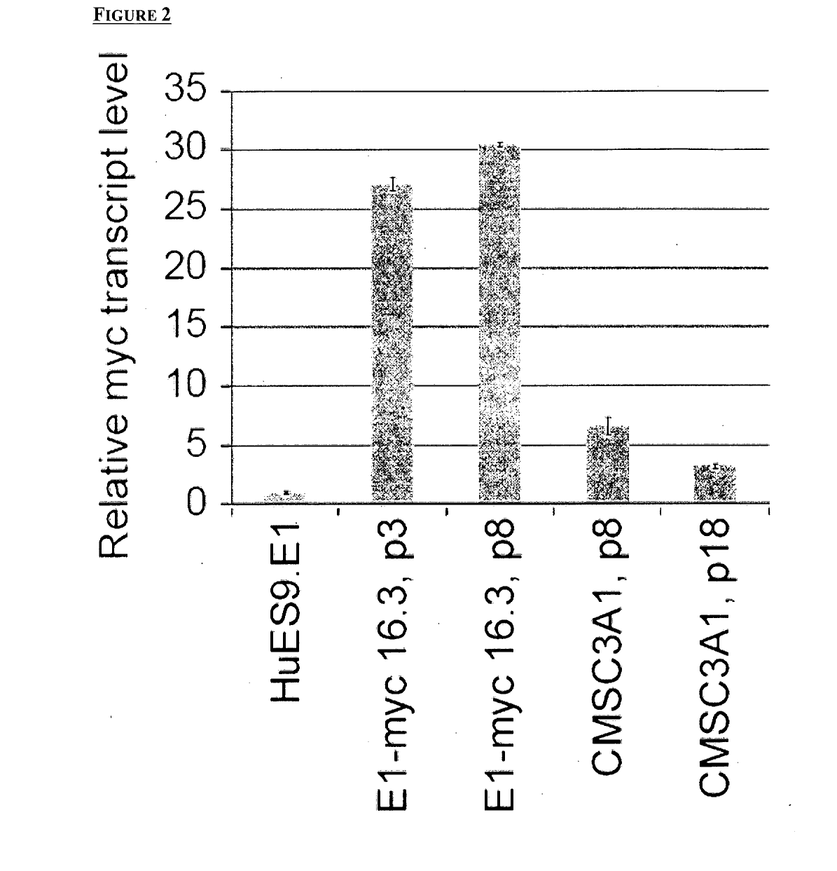

The results are shown in FIG. 2.

### Example 14

### Results—Rate of Cell Cycle

Both myc-transformed cord MSC lines, CMSC3A1 and CMSC3A3 proliferated with an average cell cycle time of about 13 hours which was longer than the 11 hours of E1myc 16.3 but shorter than HuES9.E1.

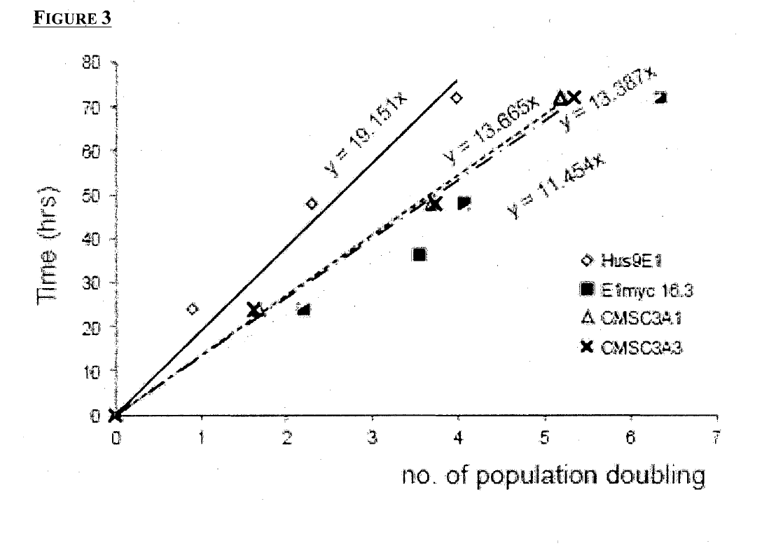

The results are shown in FIG. 3.

### Example 15

### Results—Cell Morphology

Consistent with this increased proliferation, the transformed cells were smaller and rounder in shape with prominent nuclei. They had reduced adherence to plastic culture and reduced contact inhibition at confluency so that the cells formed clusters instead of adhering to the plastic dish as a monolayer.

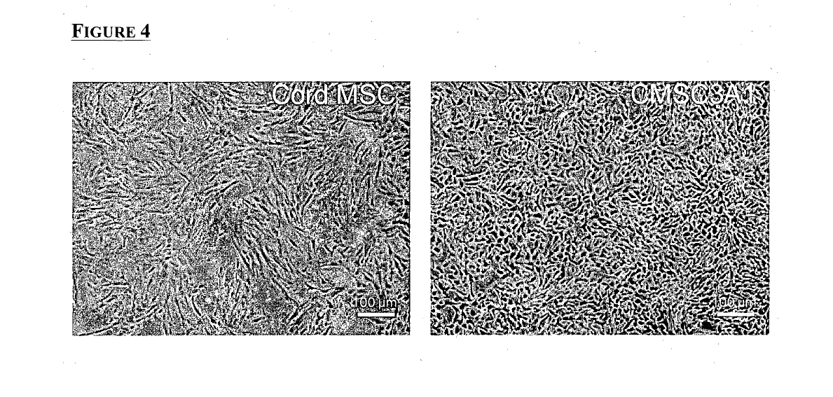

The results are shown in FIG. 4.

### Example 16

### Results—Telomerase Activity

The telomerase activity in these cells was lower than that in E1myc 16.3 but higher than that in HuES9.E1

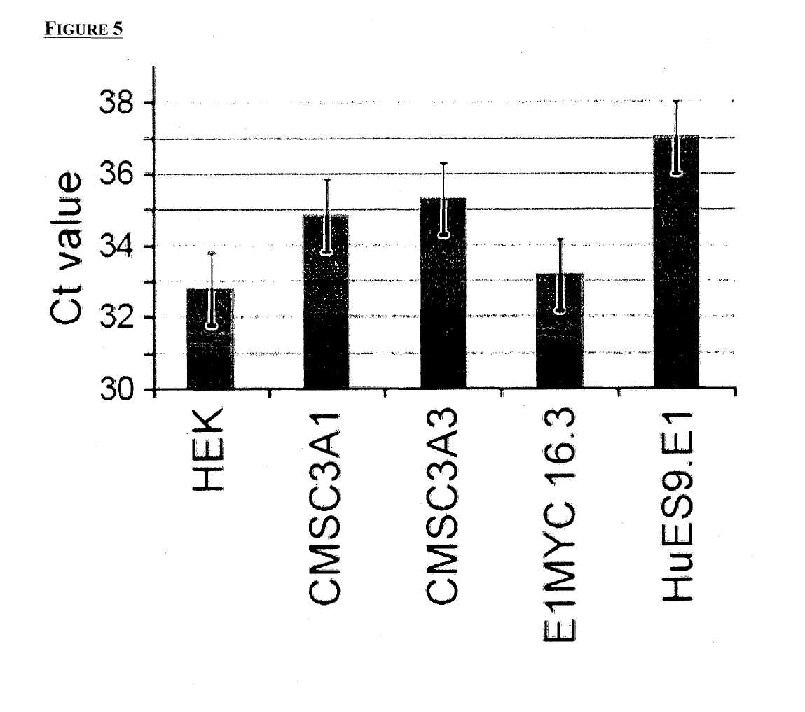

The results are shown in FIG. 5.

### Example 17

### Results—Surface Antigen Profile

The surface antigen profile of the myc-transformed cord MSCs, CMSC3A1 was quite similar to that of myc-transformed E1myc 16.3. The cells were CD29⁺, CD44⁺, CD49a⁺ CD49e⁺, CD73⁺ CD105⁺, CD166⁺, MHC I⁻, HLA-DR⁻, CD34⁻ and CD45⁻

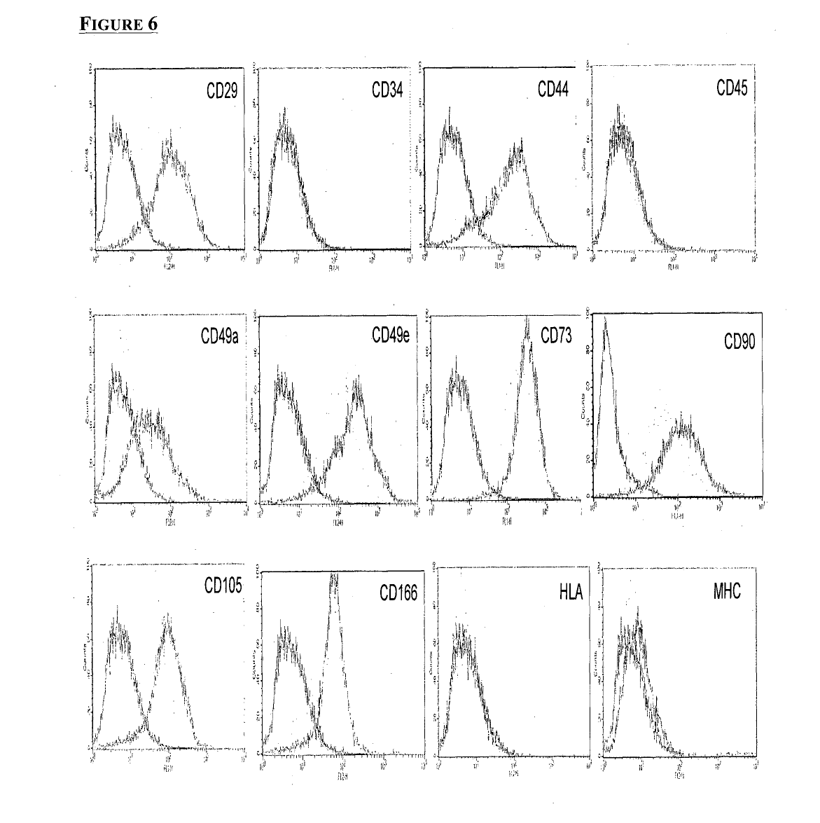

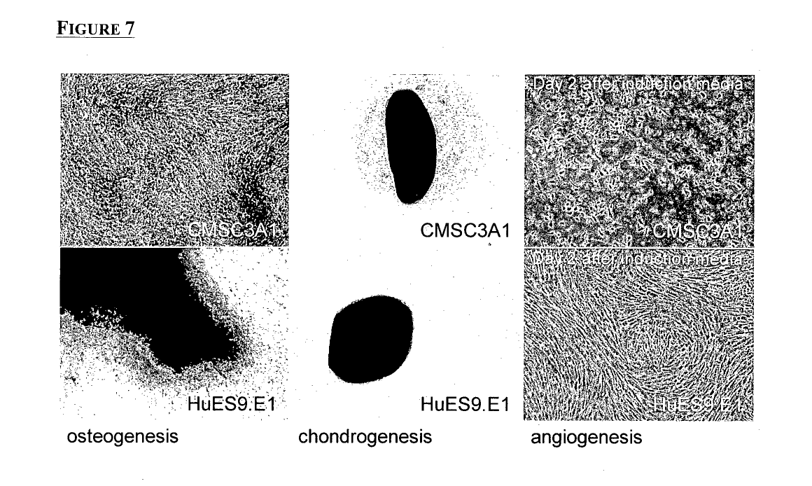

The results are shown in FIG. 6.

### Example 18

### Results—Differentiation Potential

Like myc-transformed E1myc 16.3, The in vitro differentiation potential of the myc-transformed cord MSCs, CMSC3A1 line was quite similar to that of myc-transformed E1myc 16.3 line³⁰. Unlike untransformed MSCs such as HuES9.E1 hESC-derived MSCs, both E1myc 16.3 and CMSC3A1 cells differentiated readily into chondrocytes and osteocytes but not adipocytes.

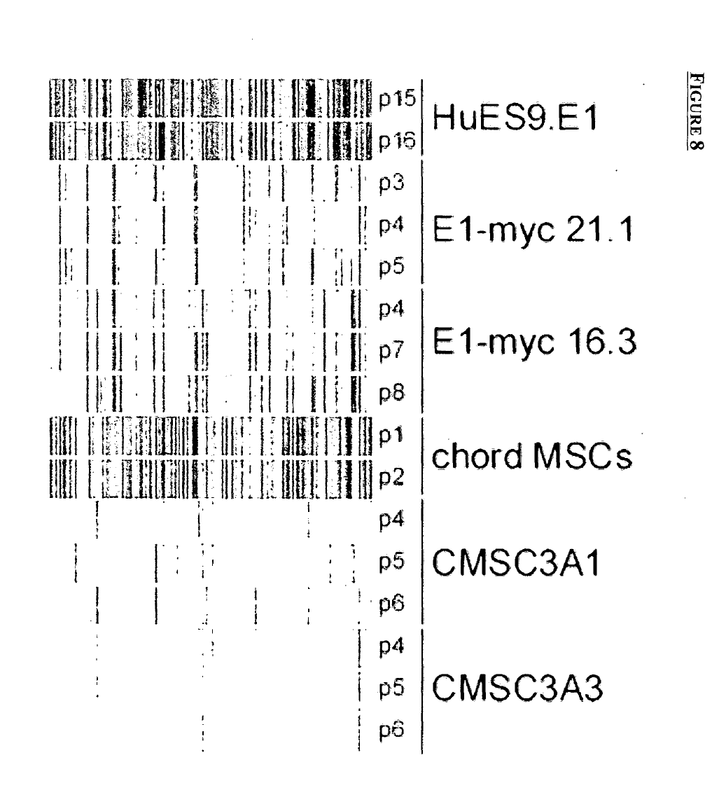

The results are shown in FIG. 7A and FIG. 7B.

During induction of adipogenesis which consisted of 4 cycles of a 6-day treatment of 3 days' exposure to induction medium and 3 days' exposure to maintenance medium, most CMSC3A1 cells like E1myc 16.3 died during exposure to the induction medium (FIG. 7C). These observations suggested that myc-transformed cord MSCs cannot undergo adipogenic differentiation which is a defining property of MSCs.

### Example 19

### Results—Gene Expression Profile

Genome-wide gene expression profiles of E1-myc 16.3, CMSC3A1, HuES9.E1 and cord-derived MSCs were determined by microarray hybridisation and assessed for the relatedness between the cell types. The myc transformed cell lines, E1-myc 16.3, CMSC3A1 had a highly similar expression profile with correlation coefficient of 0.95.

The results are shown in FIG. 8.

In contrast, E1-myc 16.3 and its parental HuES9.E1 had a correlation coefficient of 0.92 while CMSC3A3 and its parental cord-derived MSCs had a correlation coefficient of 0.92.

### Example 20

### Results—Isolation of Exosomes from Culture Medium Conditioned by CMSC 3A1

We had previously demonstrated that exosomes secreted by ESC-derived MSCs and their myc-transformed progeny were protective in a mouse model of myocardial ischemia and reperfusion injury^{27,30,35}. To test if transformed cord MSCS also produced similar exosomes, CMSC 3A1 were grown in a chemically defined medium, the conditioned culture medium (CM) were harvested and exosomes purified as previously described^{34,35}. The HPLC protein profile of the CM was similar to that of CM from ESC-derived MSCs and their myc-transformed progeny³⁵ with the fastest eluting fraction having a retention time of about 12 minutes.

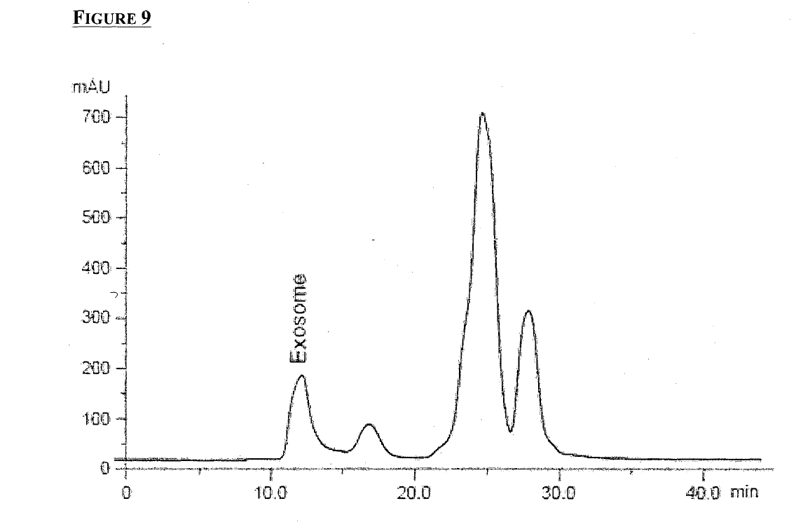

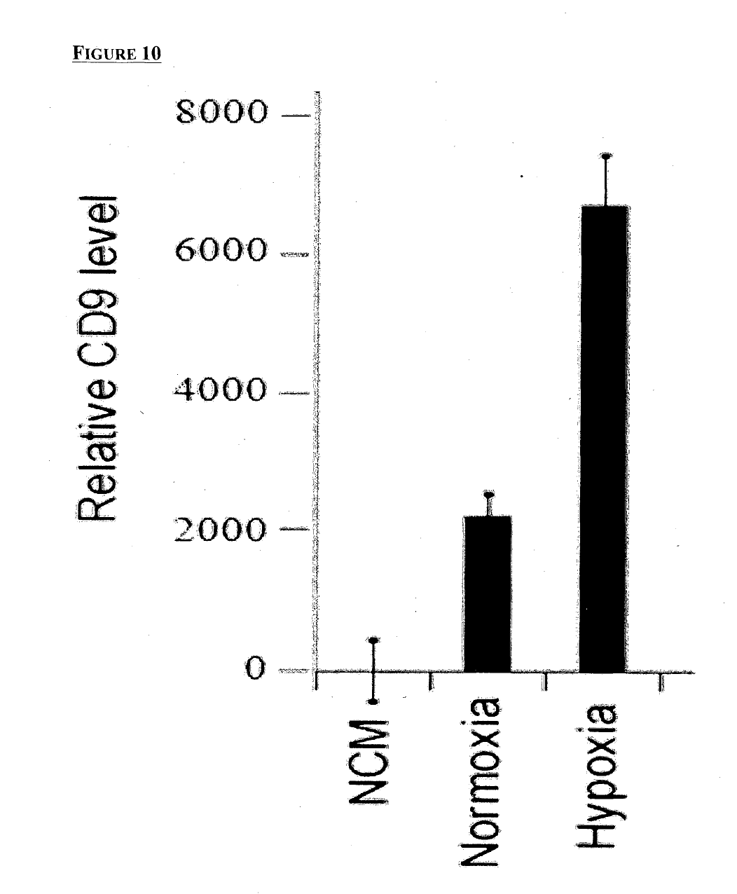

The results are shown in FIG. 9.

Dynamic light scattering analysis of this peak revealed the presence of particles that were within a hydrodynamic radius range of 50-65 ηm. Western blot analysis of this peak also revealed the presence of exosome-associated proteins such as CD9 and CD81 (data not shown).

### Example 21

### Results—Enhanced Exosome Production Under Hypoxic Culture Condition

To assess the effect of hypoxia on exosome production, CMSC 3A1 cells were grown in normoxia (atmospheric oxygen) and hypoxia (1% oxygen). The level of exosomes in the media conditioned by cells in hypoxia was about three times higher that conditioned by cells in normoxia.

The results are shown in FIG. 10.

### Example 22

### Results—Cardioprotection by CMSC3A1 Exosomes

HPLC-purified exosomes from either E1-myc 21.1 or E1-myc 16.3 was administered to the mouse model of myocardial ischemia-reperfusion injury at a dosage of 0.3 μg per mouse.

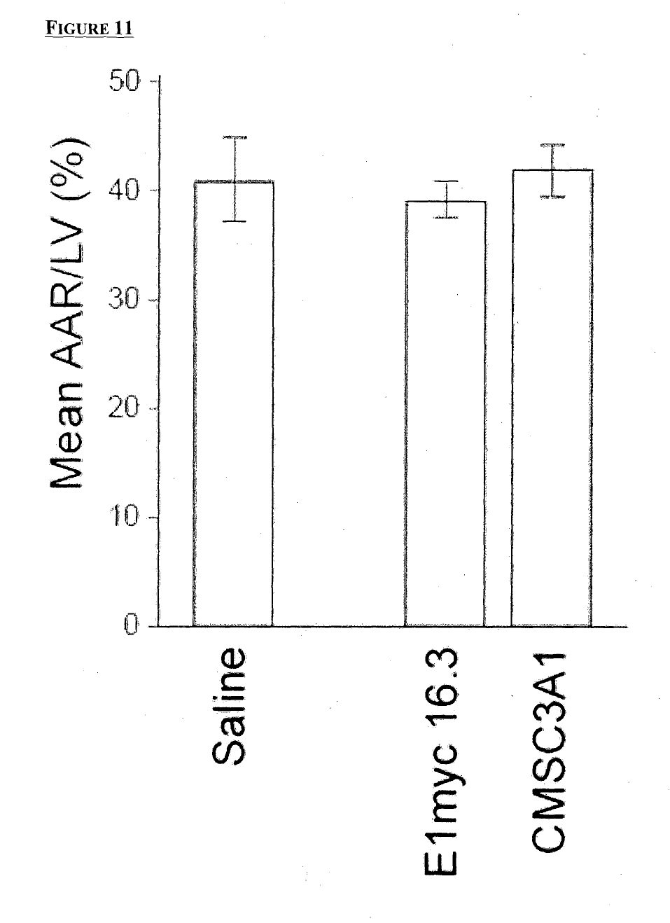

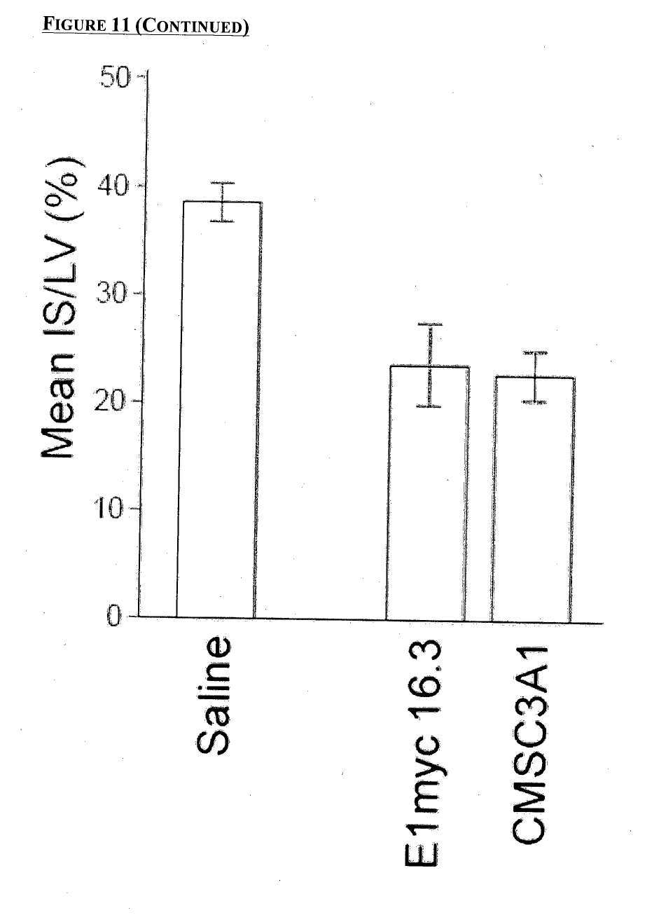

The results are shown in FIG. 11.

The area at risk (AAR) as a percentage of left ventricular (LV) area in CMSC3A1 exosome, E1-myc 16.3 exosome or the saline-treated control group was similar. The relative infarct size (IS/AAR) in mice treated with E1-myc 21.1 exosome or E1-myc 16.3 exosome was 22.6±4.5%, and 19.8±2.9%, respectively and their relative infarct sizes were significantly lower than the relative infarct size of 38.5±5.6% in saline-treated mice (p<0.002 and p<0.001, respectively.

### Example 23

### Results—Exosomes Secreted by Myc-Immortalized Cord MSCs are Different from that Secreted by ESC-Derived MSCs

Exosomes secreted by Myc-immortalized cord MSCs and Myc-immortalized ESC-derived MSCs, E1-myc were assessed for the presence of GM1 gangliosides or phosphatidylserine on membrane surface by testing their affinity for cholera toxin B chain (CTB) and Annexin V (AV), respectively.

Briefly, 1 μg of exosomes from either myc immortalized human ESC-derived MSCs (E1-myc) or myc immortalized cord derived MSCs (cord 1/cord2) were separately incubated with cholera toxin B chain (CTB) and Annexin (AV). The bound exosomes are then immobilised using solid support conjugated to either the CTB or AV e.g. magnetic beads. The bound exosomes were probed with mouse anti-human CD9 antibody followed by a HRP-conjugated donkey anti-mouse IgG antibody. All antibodies were purchased from Santa Cruz. The complex was then incubated with a HRPsubstrate. The relative amount of exosomes bound by CTB or AV was determined by HRP activity and the ratio of AV to CTB was calculated.

The ratio of AV+ to CTB+ exosomes secreted by Myc-immortalized cord MSCs (Cord 1 and Cord2) is 10-15 times higher than that secreted by Myc-immortalized ESC-derived MSCs (E1-myc).

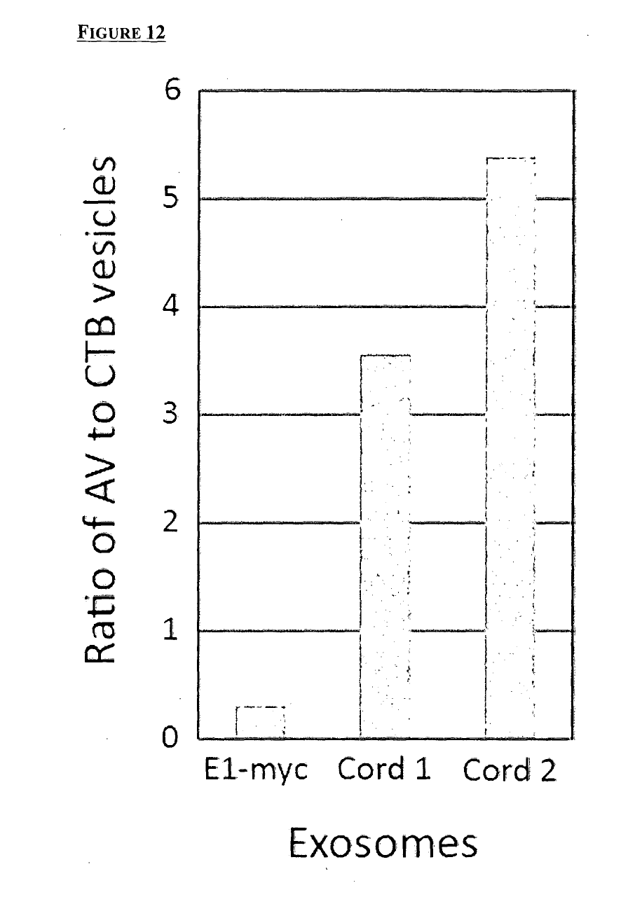

The results are shown in FIG. 12. This demonstrates that exosomes secreted by myc-immortalized cord MSCs are different from that secreted by Myc-immortalized ESC-derived MSCs.

### Example 24

### Results—Discussion

This report describes the transformation of human umbilical-derived MSCs by over-expression of a c-myc gene. This transformation enabled the cells to bypass senescence, increase telomerase activity and enhance proliferation. Generally, genome-wide gene expression between the transformed cells versus their parental cells was conserved with a correlation coefficient of 0.92. However, we observed a higher correlation of 0.95 between myc-transformed MSCs that were derived from either human ESCs (i.e. E1-myc 21.1 and E1-myc 16.3) or human umbilical cord (i.e. CMSC3A1 and CMSC3A3). The transformed cells also have the characteristic surface antigen profile of MSC: CD29⁺, CD44⁺, CD49a⁺ CD49e⁺, CD105⁺, CD166⁺, MHC I⁻, HLA-DR⁻, CD34⁻ and CD45⁻. However they have an altered MSC phenotype such as a reduced adherence to plastic and a failure to undergo adipogenesis. Therefore, in contrast to a previous report³¹ that observed no fundamental changes in MSC properties after myc transformation but consistent with our previous observations³⁰, myc transformation induced fundamental changes in MSCs such that they are technically not MSCs. Notwithstanding this, the myc-transformed cells continued to secrete exosomes that could reduce infarct size in a mouse model of ischemia/reperfusion injury. We also observed that the cells produced more exosomes under hypoxia.

### REFERENCES

1. Le Blanc, K. & Pittenger, M. Mesenchymal stem cells: progress toward promise. Cytotherapy 7, 36-45 (2005).

2. Minguell, J. J. & Erices, A. Mesenchymal stem cells and the treatment of cardiac disease. Exp Biol Med (Maywood) 231, 39-49 (2006).

3. Schuleri, K. H., Boyle, A. J. & Hare, J. M. Mesenchymal stem cells for cardiac regenerative therapy. Handbook of experimental pharmacology, 195-218 (2007).

4. Abdel-Latif, A., et al. Adult bone marrow-derived cells for cardiac repair: a systematic review and meta-analysis. Archives of internal medicine 167, 989-997 (2007).

5. Mazhari, R. & Hare, J. M. Advances in cell-based therapy for structural heart disease. Prog Cardiovasc Dis 49, 387-395 (2007).

6. Ohnishi, S. & Nagaya, N. Prepare cells to repair the heart: mesenchymal stem cells for the treatment of heart failure. American journal of nephrology 27, 301-307 (2007).

7. Behfar, A. & Terzic, A. Optimizing adult mesenchymal stem cells for heart repair. J Mol Cell Cardiol 42, 283-284 (2007).

8. Atsma, D. E., Fibbe, W. E. & Rabelink, T. J. Opportunities and challenges for mesenchymal stem cell-mediated heart repair. Curr Opin Lipidol 18, 645-649 (2007).

9. Gnecchi, M., et al. Paracrine action accounts for marked protection of ischemic heart by Akt-modified mesenchymal stem cells. Nat Med 11, 367-368 (2005).

10. Caplan, A. I. & Dennis, J. E. Mesenchymal stem cells as trophic mediators. J Cell Biochem 98, 1076-1084 (2006).

11. Kinnaird, T., et al. Local delivery of marrow-derived stromal cells augments collateral perfusion through paracrine mechanisms. Circulation 109, 1543-1549 (2004).

12. Leedham, S. J., Brittan, M., McDonald, S. A. & Wright, N. A. Intestinal stem cells. J Cell Mol Med 9, 11-24 (2005).

13. Togel, F., et al. Administered mesenchymal stem cells protect against ischemic acute renal failure through differentiation-independent mechanisms. Am J Physiol Renal Physiol 289, F31-42 (2005).

14. Patschan, D., Plotkin, M. & Goligorsky, M. S. Therapeutic use of stem and endothelial progenitor cells in acute renal injury: ca ira. Curr Opin Pharmacol 6, 176-183 (2006).

15. Miyahara, Y., et al. Monolayered mesenchymal stem cells repair scarred myocardium after myocardial infarction. Nat Med 12, 459-465 (2006).

16. Gnecchi, M., et al. Evidence supporting paracrine hypothesis for Akt-modified mesenchymal stem cell-mediated cardiac protection and functional improvement. Faseb J 20, 661-669 (2006).

17. Mayer, H., et al. Vascular endothelial growth factor (VEGF-A) expression in human mesenchymal stem cells: autocrine and paracrine role on osteoblastic and endothelial differentiation. J Cell Biochem 95, 827-839 (2005).

18. Nakagami, H., et al. Novel autologous cell therapy in ischemic limb disease through growth factor secretion by cultured adipose tissue-derived stromal cells. Arterioscler Thromb Vasc Biol 25, 2542-2547 (2005).

19. Van Overstraeten-Schlogel, N., Beguin, Y. & Gothot, A. Role of stromal-derived factor-1 in the hematopoietic-supporting activity of human mesenchymal stem cells. Eur J Haematol 76, 488-493 (2006).

20. Cheng, L., Qasba, P., Vanguri, P. & Thiede, M. A. Human mesenchymal stem cells support megakaryocyte and pro-platelet formation from CD34(+) hematopoietic progenitor cells. J Cell Physiol 184, 58-69 (2000).

21. Caplan, A. I. & Dennis, J. E. Mesenchymal stem cells as trophic mediators. J Cell Biochem (2006).

22. Liu, C. H. & Hwang, S. M. Cytokine interactions in mesenchymal stem cells from cord blood. Cytokine 32, 270-279 (2005).

23. Pittenger, M. F. & Martin, B. J. Mesenchymal stem cells and their potential as cardiac therapeutics. Circ Res 95, 9-20 (2004).

24. Lian, Q., et al. Derivation of clinically compliant MSCs from CD105+, CD24− differentiated human ESCs. Stem Cells 25, 425-436 (2007).

25. Lai, R. C., et al. Derivation and characterization of human fetal MSCs: An alternative cell source for large-scale production of cardioprotective microparticles. J Mol Cell Cardiol.

26. Lai, R. C., et al. Exosome secreted by MSC reduces myocardial ischemia/reperfusion injury Stem Cell Res (2010).

27. Timmers, L., et al. Reduction of myocardial infarct size by human mesenchymal stem cell conditioned medium. Stem Cell Research 1, 129-137 (2008).

28. Chen, T. S., et al. Mesenchymal stem cell secretes microparticles enriched in pre-microRNAs. Nucleic Acids Res 38, 215-224 (2010).

29. Sun, D., et al. A Novel Nanoparticle Drug Delivery System: The Anti-inflammatory Activity of Curcumin Is Enhanced When Encapsulated in Exosomes. Mol Ther 18, 1606-1614 (2010).

30. Chen, T. S., et al. Enabling a robust scalable manufacturing process for therapeutic exosomes through oncogenic immortalization of human ESC-derived MSCs. J Transl Med 9, 47 (2011).

31. Nagai, A., et al. Multilineage Potential of Stable Human Mesenchymal Stem Cell Line Derived from Fetal Marrow. PLoS ONE 2, e1272 (2007).

32. Kitamura, T., et al. Retrovirus-mediated gene transfer and expression cloning: powerful tools in functional genomics. Exp Hematol 31, 1007-1014 (2003).

33. Wege, H., Chui, M. S., Le, H. T., Tran, J. M. & Zern, M. A. SYBR Green real-time telomeric repeat amplification protocol for the rapid quantification of telomerase activity. Nucleic Acids Res 31, E3-3 (2003).

34. Sze, S. K., et al. Elucidating the secretion proteome of human embryonic stem

[symbol]

35. Lai, R. C., et al. Exosome secreted by MSC reduces myocardial ischemia/reperfusion injury. Stem Cell Res 4, 214-222 (2010).

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method comprising the steps of:
(a) providing an umbilical mesenchymal stem cell (MSC); and
(b) culturing the umbilical mesenchymal stem cell in a cell culture medium under hypoxic conditions.

2. The method of claim 1, wherein the umbilical mesenchymal stem cell comprises a transformed umbilical mesenchymal stem cell.

3. The method of claim 2, wherein the umbilical mesenchymal stem cell or an ancestor thereof is transformed by introducing an oncogene.

4. The method of claim 3, wherein said oncogene is c-myc.

5. The method of claim 1, wherein the hypoxic conditions comprise 10% or less oxygen.

6. A method of producing an umbilical mesenchymal stem cell conditioned medium (MSC-CM), the method comprising:
(a) culturing the umbilical mesenchymal stem cell in a cell culture medium under hypoxic conditions to condition it; and
(b) separating the conditioned cell culture medium from the umbilical mesenchymal stem cell;
thereby producing an umbilical mesenchymal stem cell conditioned medium (MSC-CM).

7. The method of claim 1, further comprising isolating an exosome from the cell culture medium.

8. The method of claim 6, further comprising the steps of:
(a) concentrating the umbilical mesenchymal stem cell conditioned medium;
(b) subjecting the concentrated umbilical mesenchymal stem cell conditioned medium to size exclusion chromatography; and
(c) selecting UV absorbant fractions that exhibit dynamic light scattering.

9. The method of claim 8, in which step (b) comprises size exclusion chromatography on a TSK Guard column SWXL, 6×40 mm or a TSK gel G4000 SWXL, 7.8×300 mm column, and step (c) comprises collecting fractions which elute with a retention time of 11-13 minutes.

10. The method of claim 1, wherein said umbilical mesenchymal stem cell cultured under hypoxic conditions produces 150% or more exosomes than an umbilical mesenchymal stem cell cultured under normoxic conditions.

11. A method of preparing a pharmaceutical composition, the method comprising admixing an umbilical mesenchymal stem cell conditioned medium (MSC-CM) prepared by the method of claim 1, or an exosome isolated therefrom, with a pharmaceutically acceptable carrier or diluent.

12. A pharmaceutical composition prepared by the method of claim 11.

13. A composition comprising an umbilical mesenchymal stem cell conditioned medium prepared by the method of claim 6, or an exosome isolated therefrom.

14. The composition of claim 13 or a pharmaceutical composition prepared therefrom, wherein the conditioned medium, exosome or pharmaceutical composition comprises at least one biological property of a umbilical mesenchymal stem cell selected from the group consisting of cardioprotection, reduction of infarct size as assayed in a mouse or pig model of myocardial ischemia and reperfusion injury, and reduction of oxidative stress as assayed in an in vitro assay of hydrogen peroxide (H₂0₂)-induced cell death.

15. A method of treating a disease in an individual, the method comprising:
(a) culturing an umbilical mesenchymal stem cell under hypoxic conditions;
(b) obtaining an umbilical mesenchymal stem cell conditioned medium (MSC-CM) or an exosome from a culture of step (a); and
(c) administering the umbilical mesenchymal stem cell conditioned medium (MSC-CM) or exosome obtained in step (b) to an individual in need thereof.

16. The method of claim 15, wherein step (b) comprises
(i) concentrating the umbilical mesenchymal stem cell conditioned medium;
(ii) subjecting the concentrated umbilical mesenchymal stem cell conditioned medium to size exclusion chromatography; and
(iii) selecting UV absorbant fractions that exhibit dynamic light scattering.

17. An umbilical mesenchymal stem cell obtained from an umbilical tissue, a descendent of such an umbilical mesenchymal stem cell, a cell culture or a cell line comprising either, which is or has been cultured under hypoxic conditions, and which produces 150% or more exosomes than an umbilical mesenchymal stem cell, descendent, cell culture or cell line cultured under normoxic conditions.

18. An umbilical mesenchymal stem cell, descendent, cell culture or cell line of claim 17, into which, or into an ancestor of which, an oncogene has been introduced to thereby transform it.

19. The umbilical mesenchymal stem cell, descendent, cell culture or cell line of claim 18, in which said oncogene comprises c-myc.

20. A conditioned medium or an exosome obtained from an umbilical mesenchymal stem cell, descendent, cell culture or cell line of claim 17.
